(19) United States
(12) Patent Application Publication
(10) Pub. No.: US 2022/0348876 A1
(43) Pub. Date: Nov. 3, 2022

(54) METABOLISM GUIDES DEFINITIVE LINEAGE SPECIFICATION DURING ENDOTHELIAL TO HEMATOPOIETIC TRANSITION

(71) Applicant: Amniotics AB, Lund (SE)

(72) Inventors: Niels-Bjarne Woods, Furulund (SE); Leal Oburoglu, Lund (SE)

(21) Appl. No.: 17/756,618

(22) PCT Filed: Nov. 27, 2020
(86) PCT No.: PCT/SE2020/051139

§ 371 Date: May 27, 2022

(30) Foreign Application Priority Data
Nov. 28, 2019 (SE) 1930385-8
Feb. 12, 2020 (SE) 2030046-3

(51) Int. Cl. C12N 5/0789

(52) U.S. Cl. CPC C12N 5/0647; C12N 2502/02; C12N 2506/45; C12N 2500/40; C12N 2500/32; C12N 2501/125; C12N 2501/26; C12N 2501/2302; C12N 2501/2307; C12N 2501/2315

(57) ABSTRACT

Methods of generating definitive hematopoietic cells from source cells including at least one of: differentiating iPS cells, cells directly reprogrammed to pre-cursors of hematopoietic cells, cells directly reprogrammed to definitive hematopoietic cells, and adult or neonatal hematopoietic cells from bone marrow, cord blood, placenta, or mobilized peripheral blood, the method including using a metabolic regulator to activate a tricarboxylic acid cycle of the source cells. Other methods relate to generating primitive hematopoietic cells from source cells including at least one of: differentiating iPS cells, cells directly reprogrammed to pre-cursors of hematopoietic cells, cells directly reprogrammed to definitive hematopoietic cells, and adult or neonatal hematopoietic cells from bone marrow, cord blood, placenta, or mobilized peripheral blood, the method including using a metabolic regulator to inhibit a tricarboxylic acid cycle of the source cells. Some aspects relate to a metabolic regulator for activation of a tricarboxylic acid cycle of source cells for the production of definitive or primitive hematopoietic cells.

### INCORPORATION BY REFERENCE TO ANY PRIORITY APPLICATIONS

Any and all applications for which a foreign or domestic priority claim is identified in the Application Data Sheet as filed with the present application are hereby incorporated by reference under 37 CFR 1.57.

### BACKGROUND

### Field of the Invention

A method of generating definitive hematopoietic cells from source cells, the definitive hematopoietic cells including at least one of: differentiating iPS cells, cells directly reprogrammed to pre-cursors of hematopoietic cells, cells directly reprogrammed to definitive hematopoietic cells, and adult or neonatal hematopoietic cells from bone marrow, cord blood, placenta, or mobilized peripheral blood, the method including using a metabolic regulator to activate a tricarboxylic acid cycle of the source cells.

### Description of the Related Art

In the developing embryo, primitive hematopoiesis gives rise to erythrocytes, megakaryocytes and macrophages in the blood islands of the yolk sac (YS) (Palis, J. et al. Development 126, 5073-5084 (1999)). Next, a definitive wave of hematopoiesis produces more mature erythro-myeloid (Palis, J. et al. Development 126, 5073-5084 (1999)) and lymphoid (Yoder, M. C. et al. Immunity 7, 335-344 (1997); and Böiers, C. et al. Cell Stem Cell 13, 535-548 (2013)) progenitors. Around Carnegie stage (CS) 12-13, hematopoietic stem cells (HSCs) emerge in the aorta-gonad-mesonephros (AGM) region through a second definitive hematopoietic wave (Medvinsky, A. & Dzierzak, E. Cell 86, 897-906 (1996); and Ivanovs, A. et al. J Exp Med 208, 2417-2427 (2011)). Primitive erythrocytes, erythro-myeloid progenitors (EMPs) and HSCs derive from a hemogenic endothelial (HE) cell (Lancrin, C. et al. Nature 457, 892-895 (2009); Frame, J. M. et al. STEM CELLS 34, 431-444 (2016) and Stefanska, M. et al. Sci Rep 7, 1-10 (2017)) by a process known as endothelial to hematopoietic transition (EHT) (Boisset, J.-C. et al. Nature 464, 116-120 (2010); and Kissa, K. & Herbomel, P. Nature 464, 112-115 (2010)). Studies on hematopoietic emergence during embryonic development have not only described EHT in spatial and temporal contexts in several animal models (Boisset, J.-C. et al. Nature 464, 116-120 (2010); and Kissa, K. & Herbomel, P. Nature 464, 112-115 (2010)) but also led to a deep understanding of the growth and transcription factors regulating this process (Chen, M. J. et al. Nature 457, 887-891 (2009); Zhou, F. et al. Nature 533, 487-492 (2016); and Swiers, G. et al. Nat Commun 4, 2924 (2013)). However, the role of metabolites and metabolic pathways in the emergence of hematopoietic cells has not been evaluated during development.

Growing evidence points to the fact that metabolic pathways can control cell fate (Oburoglu, L. et al. Cell Stem Cell 15, 169-184 (2014); Moussaieff, A. et al. Cell Metabolism 21, 392-402 (2015); and Folmes, C. D. L. et al. Cell Metabolism 14, 264-271 (2011)). Specifically, the fate of bone marrow HSCs is regulated by several metabolic pathways. The hypoxic niche of the bone marrow pushes HSCs to activate a minimal energy-providing pathway, anaerobic glycolysis, and ensures their quiescent state (Takubo, K. et al. Cell Stem Cell 12, 49-61 (2013)). HSC self-renewal and maintenance rely on fatty acid oxidation (Ito, K. et al. Nat Med 18, 1350-1358 (2012)) and differentiating HSCs switch to oxidative phosphorylation (OXPHOS) to meet their energetic requirements (Yu, W.-M. et al. Cell Stem Cell 12, 62-74 (2013); and Simsek, T. et al. Cell Stem Cell 7, 380-390 (2010)).

The EHT process has been modeled extensively in vitro using pluripotent stem cells (PSCs) and the HE intermediate which arises in this context can give rise to both primitive and definitive hematopoietic cells (Garcia-Alegria, E. et al. Stem Cell Reports 11, 1061-1074 (2018)). Many studies have focused on obtaining HE with solely definitive potential in vitro (Kennedy, M. et al. Cell Reports 2, 1722-1735 (2012); Sugimura, R. et al. Nature 545, 432-438 (2017); Ng, E. S. et al. Nature Biotechnology 34, 1168-1179 (2016); and Sturgeon, C. M. et al. Nat Biotech 32, 554-561 (2014)), in an effort to produce functional and transplantable HSCs for therapeutic use.

As EHT implicates tight-junction dissolution, gain of stem cell-like properties and leads to extensive transcriptional and phenotypic changes in the transitioning cell (Zhou, F. et al. Nature 533, 487-492 (2016); Swiers, G. et al. Nat Commun 4, 2924 (2013); and Guibentif, C. et al. Cell Reports 19, 10-19 (2017)), metabolism may contribute to regulating these processes. Previously, in animal models, the emergence of HSCs was shown to be regulated by adenosine signaling and the PKA-CREB pathway (Jing, L. et al. J Exp Med 212, 649-663 (2015); and Kim, P. G. et al. J Exp Med 212, 633-648 (2015)), which are tightly controlled by ATP levels and availability; suggesting a change in energy demand during EHT. Moreover, glucose metabolism has been shown to induce HSC emergence in zebrafish (Harris, J. M. et al. Blood 121, 2483-2493 (2013)).

As will be understood by one of skill in the art, currently there are limitations in the availability of suitably matched hematopoietic cells for transplantation or transfusion procedures required in the routine treatment of over 100 hematological diseases, malignancies, and other life-threatening indications. The current sources of hematopoietic cells and hematopoietic stem cells are limiting because they typically rely on donations by healthy individuals as part of blood drives (e.g., Red Cross) and stem cell donor registries for bone marrow, cord blood, and mobilized peripheral blood. These shortages of suitable donor blood products limit the ability to perform necessary therapies, therefore up to 30% of patients in need of hematopoietic stem cell transplantation for treatment of malignancies do not have a suitably matched donor, and a complicated infrastructure of transportation of transfusable blood cells and good will donor blood drives address shortages in supply as need varies over time and geographical area. Thus, there is a significant need for a more robust and reliable system for acquiring both hematopoietic stem cells and transfusable blood cell products.

There are also risks associated with using donor derived products, such as transmission of infections to the recipient patients, and tissue rejection complications such as graft versus host disease, both of which are potentially life-threatening. Therefore, development of an alternative source of these hematopoietic cells, with practically unlimited self-renewal ability, that could be perfectly matched to the recipient, without risk of transmission of infections, is needed.

### SUMMARY

We have determined that metabolic modulations prompt HE cells to preferentially adopt a definitive hematopoietic fate. We show a gradual and global increase in metabolism during human EHT, fueled by glucose, glutamine and pyruvate. By dissecting the use of these nutrients, we have elucidated their roles in hematopoietic lineage specification.

Some aspects relate to a method of generating definitive hematopoietic cells from source cells, the source cells including at least one of:

differentiating iPS cells,

cells directly reprogrammed to pre-cursors of hematopoietic cells,

cells directly reprogrammed to definitive hematopoietic cells, and

adult or neonatal hematopoietic cells from bone marrow, cord blood, placenta, or mobilized peripheral blood; and

the method including using a metabolic regulator to activate a tricarboxylic acid cycle of the source cells.

In some examples, the metabolic regulator inhibits Pyruvate dehydrogenase kinases (PDK).

In some examples, the metabolic regulator activates Pyruvate Dehydrogenase complexes (PDH).

In some examples, the metabolic regulator increases uptake of Pyruvate into mitochondria.

In some examples, the metabolic regulator accelerates conversion of Pyruvate to acetyl coenzyme A (Ac-CoA).

In some examples, the metabolic regulator is dichloroacetate (DCA).

In some examples, the concentration of the dichloroacetate in a culture media for the source cells is at least 30 μM.

In some examples, the DCA induces lymphoid/myeloid-biased definitive hematopoiesis.

In some examples, the metabolic regulator is an LSD1 inhibitor.

In some examples, the LSD1 inhibitor includes at least one of GSK2879552 or RO7051790.

In some examples, the LSD1 inhibitor generates definitive hematopoietic cells of the erythroid lineage.

In some examples, the metabolic regulator increases production of α-ketoglutarate.

In some examples, the metabolic regulator is glutamine.

In some examples, the metabolic regulator results in the generation of CD43+ cells from a hemogenic endothelial (HE) source cell.

In some examples, the method further includes using nucleoside triphosphates.

In some examples, the metabolic regulator is a more potent or more stable equivalent of α-ketoglutarate.

In some examples, the metabolic regulator is Dimethyl α-ketoglutarate (DMK).

In some examples, the concentration of Dimethyl α-ketoglutarate in a culture media for the differentiating iPS cells is at least 17.5 μM.

In some examples, the metabolic regulator is used in combination with Nucleosides.

In some examples, the concentration of Nucleosides is at least 0.7 mg/L.

In some examples, the Nucleosides include at least one of Cytidine, Guanosine, Uridine, Adenosine, Thymidine.

In some examples, the definitive hematopoietic cells include definitive hematopoietic stem cells.

In some examples, the definitive hematopoietic stem cells have lymphoid and/or myeloid repopulating potential.

In some examples, the definitive hematopoietic cells include definitive lymphoid and/or myeloid cells.

In some examples, the definitive lymphoid cells include at least one of T-cells, modified T-cells targeting tumor cells, B-cells, NK cells and NKT cells.

In some examples, the definitive hematopoietic cells include mast cells.

In some examples, the definitive hematopoietic cells include erythroid cells suitable for production of adult hemoglobin.

In some examples, cells directly reprogrammed to pre-cursors of hematopoietic cells include at least one of mesodermal precursor cells, hemogenic endothelium cells, and cells undergoing endothelial to hematopoietic transition.

In some examples, adult or neonatal hematopoietic cells include hematopoietic stem cells or hematopoietic progenitor cells.

Some aspects relate to a method of generating primitive hematopoietic cells from source cells, the source cells including at least one of:

differentiating iPS cells,

cells directly reprogrammed to pre-cursors of hematopoietic cells,

cells directly reprogrammed to definitive hematopoietic cells, and

adult or neonatal hematopoietic cells from bone marrow, cord blood, placenta, or mobilized peripheral blood; and

the method including using a metabolic regulator to inhibit a tricarboxylic acid cycle of the source cells.

In some examples, the metabolic regulator inhibits uptake of Pyruvate into mitochondria.

In some examples, the metabolic regulator inhibits conversion of Pyruvate to Ac-CoA.

In some examples, the metabolic regulator inhibits MPC.

In some examples, the metabolic regulator is UK5099.

In some examples, the concentration of UK5099 in a culture media for the source cells is at least 100 nM.

In some examples, the metabolic regulator inhibits PDH.

In some examples, the metabolic regulator is 1-Aminoethylphosphinic acid (1-AA).

In some examples, the concentration of 1 Aminoethylphosphinic acid in a culture media for the source cells is at least 4 μM.

Some aspects relate to a metabolic regulator for activation of a tricarboxylic acid cycle of source cells for the production of definitive hematopoietic cells.

Some aspects relate to a metabolic regulator for activation of a tricarboxylic acid cycle of source cells for the production of primitive hematopoietic cells.

### BRIEF DESCRIPTION OF THE DRAWINGS

One of skill in the art will understand that the figures below represent examples of data and diagrams showing the information as described below.

FIG. 1a is an example of data wherein iPSC-derived cells match primary human EHT populations. iPSC-derived HE, EHT and HSC-like cells were sorted, cultured for 1 day and analyzed by scRNAseq; UMAP visualization of scRNAseq data from HE, EHT and HSC-like cells are shown, colored by sorting phenotype.

FIG. 1b is an example of data wherein iPSC-derived cells match primary human EHT populations. The figure includes a heatmap showing expression levels of endothelial and hematopoietic genes in HE, EHT and HSC-like populations.

FIG. 1c is an example of data wherein iPSC-derived cells match primary human EHT populations. The UMAP showing AEC/Hem cluster cells from Carnegie stage (CS) 1333 is matched against the HE, EHT and HSC-like populations in FIG. 1a.

FIG. 1d is an example of data wherein iPSC-derived cells match primary human EHT populations. The heatmap shows expression levels of endothelial and hematopoietic genes in AEC/Hem cluster cells which have mapped to the HE, EHT and HSC-like populations as shown in FIG. 1c.

FIG. 2 is an example of data wherein Glycolysis, oxygen consumption and mitochondrial activity increase during EHT. (a) Extracellular acidification rate (ECAR) was measured in HE (n=24), EHT (n=13) and HSC-like (n=8) cells and glycolytic flux was assessed by extracellular flux analysis. Bar graphs show relative levels±s.e.m. of the indicated processes (from 7 (HE, EHT) or 3 (HSC-like) independent experiments, unpaired t-tests). (b) Dot plots showing gene expression levels of glycolytic enzymes detected by scRNAseq and based on percent expressed (size of the dots) and average level of expression (color intensity). (c) FACS-sorted HE cells were subcultured with or without 2-DG (1 mM). Subculture day 3 representative FSC-A/CD43 plots are shown (n=7, see Extended data FIG. 3d for bar graphs). (d) Subculture day 3 representative GPA/CD43 plots and subculture day 6 CD45/CD43 plots are shown (n=6 and n=5, see Extended data FIG. 3e for bar graphs). (e) Subculture day 3 CellTrace Violet (CTV) fluorescence was assessed by flow cytometry (representative of n=4). (f) 2-NBDG uptake was measured by flow cytometry on day 10 for HE, EHT and HSC-like cells and mean MFI levels±s.e.m. are shown (n=4, paired t-tests). (g) Oxygen consumption rate (OCR) was measured in HE and EHT cells (n=7) and oxidative phosphorylation was assessed by extracellular flux analysis. Bar graphs show relative levels±s.e.m. of the indicated processes (from 3 independent experiments; unpaired t-tests). (h) TMRE fluorescence, with or without 100 μM FCCP treatment, was measured by flow cytometry on day 10 for HE, EHT and HSC-like cells and MFI—MFI FMO levels±s.e.m. relative to HE are shown (n=5, paired t-tests). (i) Basal OCR was measured in day 10 HE (n=6), EHT (n=5) and HSC-like (n=4) cells and bar graphs show mean levels±s.e.m. relative to HE (paired t-tests). (j) Live cell imaging of HE and HSC-like cells stained with TMRE (red) at day 3 of subculture. Representative merged brightfield/TMRE and TMRE images are shown. Scale bars, 100 μm. Bar graphs show means of TMRE staining intensity from all replicate wells across all experiments (HE spindle, n=9; HE round, n=9; HSC-like, n=6, Kruskal-Wallis test with multiple comparisons). (k) Dot plots showing gene expression levels of TCA cycle enzymes detected by scRNAseq and based on percent expressed (size of the dots) and average level of expression (color intensity). ns, not significant, *p<0.05, **p<0.01, ***p<0.001, ****p<0.0001.

FIG. 3 is an example of data wherein hematopoietic specification of HE relies on glutamine catabolism. FACS-sorted HE cells were subcultured in glutamine-free medium with the indicated compounds. (a) Subculture day 3 representative plots for FSC-A/CD43 and CD34/CD43 are shown (n=3, see FIG. 11, g for bar graphs). (b) Subculture day 3 CellTrace Violet (CTV) fluorescence was assessed by flow cytometry (n=4, see FIG. 11, h for bar graphs). (c) Representative day 3 CTV plot is shown for GPA+ (in orange) and CD45+ (in blue) populations deriving from HE cells (n=6, see FIG. 11, i for graph). (d) Percentages of cells expressing GPA or CD45 in the CD43+ population±s.e.m. at day 6 of subculture are depicted (Control, n=7; DMK, n=6; DMK+nucl, n=5; DMK+nucl+NEAAs, n=3; paired t-tests with the controls, see Extended data FIG. 5j for plots). (e) Percentages of CD45+CD56+±s.e.m. cells obtained following 35-day co-culture of 3-day subcultured HE cells with OP9-DL1 stroma. During the 3-day subculture, HE cells were treated with the indicated compounds. (Control and DMK, n=5; −GLN, n=4; −GLN+nucl and DMK+nucl, n=3, see Extended data FIG. 5k for plots). ns, not significant, *p<0.05, **p<0.01.

FIG. 4 is an example of data wherein increasing pyruvate flux into mitochondria in HE cells favors a definitive hematopoietic fate. (a) Pyruvate is transported into mitochondria via the mitochondrial pyruvate carrier (MPC, inhibitor: UK5099) and converted to acetyl-coA by pyruvate dehydrogenase complex (PDH, inhibitor: 1-AA). Pyruvate dehydrogenase kinases (PDKs, inhibitor: DCA) negatively regulate PDH activity. (b-e) FACS-sorted HE cells were subcultured with or without UK5099 (10 μM) or DCA (3 mM). Subculture day 3 representative GPA/CD43 plots (b, d) and subculture day 6 representative CD45/CD43 plots (c, e) are shown (see FIG. 12, a, f, k and m for corresponding bar graphs). (f) Ratio of CFU-E to CFU-G, M, GM colonies relative to the control condition obtained from HE cells subcultured with the indicated compounds for 6 days, see Extended data FIG. 6r for percentages (n=5, paired t-test). (g) Fold change in the expression of HBE1 or HBG1-2 transcripts normalized to KLF1 in CFUs obtained from HE cells treated with UK5099 (10 μM) or DCA (3 mM) relative to non-treated cells. (h) Percentages of CD45+CD56+±s.e.m. cells obtained following 35-day co-culture of 3-day subcultured HE cells with OP9-DL1 stroma. During the 3-day subculture, HE cells were treated with the indicated compounds. (n=3, one-way ANOVA test, see FIG. 12, u for plots). (i-k) Pregnant mice were injected with UK5099 or DCA at E9.5 and fetal livers were analyzed at E14.5 by flow cytometry. FL, fetal liver. Levels of LT-HSCs (i), T and B cells (j) as percentages in fetal liver are shown for control (n=10), UK5099-treated (n=14) and DCA-treated (n=16) conditions (one-way ANOVA test). (k) The ratio of BFU-E to CFU-GM colonies obtained from sorted LT-HSCs are shown (see also data in FIG. 13, e) (one-way ANOVA test). CFU, colony forming unit; BFU, burst forming unit; E, erythroid; M, macrophage; G, granulocyte. (l-p) HE cells co-cultured with OP9-DL1 stroma were treated with DCA for 3 days and transplanted into irradiated NSG mice. Bone marrow (BM) and thymi were harvested on week 12. (l) The percentages±s.e.m. of human CD4+CD8+ double positive thymocytes in huCD45+ cells from the thymus are shown (Control, n=6; DCA, n=7; unpaired t tests). Percentages±s.e.m. of human B cells (m), CLPs (n) and CD11b+ myeloid cells (p) in huCD45+ cells from the BM are shown (Control, n=6; DCA, n=7; unpaired t tests). (o) Percentages±s.e.m. of CD11b+ myeloid cells in huCD45+ cells from PB at week 8 are shown (Control, n=6; DCA, n=6; unpaired t test). ns, not significant, *p<0.05, **p<0.01, ***p<0.001.

FIG. 5 is an example of data wherein modulation of pyruvate catabolism affects HE commitment at the single-cell level. (a) Control, UK5099-treated and DCA-treated HE cells were visualized together by UMAP and divided into 7 clusters. (b) Heatmap showing scRNAseq data of endothelial or hematopoietic genes expressed in the 7 clusters. (c) Clusters 6 (559 cells) and 7 (280 cells) were assessed independently and dot plots show expression levels of the indicated genes detected by scRNAseq and based on percent expressed (size of the dots) and average level of expression (color intensity). (d) Dot plots show expression levels of the indicated hematopoietic transcription factors in clusters 6 and 7 for HE ctrl, HE+UK5099 and HE+DCA conditions, detected by scRNAseq and based on percent expressed (size of the dots) and average level of expression (color intensity).

FIG. 6 is an example of data wherein pyruvate catabolism affects EHT via distinct mechanisms. (a) FACS-sorted HE cells were subcultured with the indicated compounds and day 3 CD43+GPA+ cell frequencies±s.e.m. relative to the control are shown (n=4, one-way ANOVA test). (b) HE cells were transduced with shScrambled (shScr) or shLSD1 with or without UK5099 (10 μM) the day after the sort and day 3 CD43+/GPA+ cell frequencies±s.e.m. relative to shScr are presented (n=3, one-way ANOVA test). (c) Acetyl-coA can be a precursor for lipid biosynthesis via ACC (inhibitor: CP-640186 or CP) or for the mevalonate pathway/cholesterol biosynthesis via HMGCR (inhibitor: Atorvastatin or Ato). (d) FACS-sorted HE cells were subcultured with CP (5 μM), DCA (3 mM) or both and day 6 CD43+CD45+ cell frequencies±s.e.m. relative to the control are shown (n=4, one-way ANOVA test). (e) Cholesterol content in HE cells were measured at day 2 of treatment by filipin III staining (n=3, paired t test). (f) FACS-sorted HE cells were subcultured with Ato (0.5 μM), DCA (3 mM) or both and day 3 CD43+CD45+ cell frequencies±s.e.m. relative to the control are shown (n=3 for control/DCA, n=2 with 2 technical replicates for Ato/Ato+DCA, one-way ANOVA test). (g) Glycolysis is essential for hematopoietic differentiation of HE cells and inhibiting pyruvate entry into mitochondria (via UK5099 or shMPC1/2) favors a primitive erythroid fate. Increasing pyruvate flux into mitochondria via DCA amplifies acetyl-coA production which fuels cholesterol biosynthesis and promotes definitive hematopoietic differentiation of HE cells.

FIG. 7 is an example of data with generation and characteristics of EHT populations of interest. (a) Schematic of the hematopoietic differentiation system. Following embryoid body setup, BMP4, Activin A, CHIR99021, VEGF and hematopoietic cytokines were added sequentially to induce HE cell formation and EHT. Cells of interest were sorted at day 8 of the protocol. (b) Sorting strategy for obtaining pure HE, EHT and HSC-like cell populations. At day 8 of differentiation, representative plots show the level of CD34+ cells following magnetic bead enrichment, separation on the basis of CD43 expression and further gating on CXCR4-CD73− and CD90+VEcad+ for HE and EHT cells; and CD90+CD38− for HSC-like cells. (c-d) Pseudotime analysis of EHT populations taking a G0 (c) or S/G2M (d) path and corresponding bar graphs showing abundance of populations. (e) scCoGAPS mapping of cord blood CD34+ cells (CB HSC) to the EHT dataset and violin plot showing pattern weights. (f-g) scCoGAPS mapping of the EHT dataset to the human CS 13 dorsal aorta dataset (f) and vice versa (g), with plots showing colocalization of populations.

FIG. 8 is an example of data with validation of the hematopoietic potential of HE and EHT cells. (a-c) Sorted HE and EHT cells were subcultured for 6 days (representative of n=5). The levels of CD43 and CD34 markers (a) and the levels of CD43, GPA and CD45 markers (c) were assessed at subculture days 3 and 6. (b) Representative pictures of the wells were taken every day during HE and EHT subculture. Scale bars, 100 μm. (d) Expression of globin genes in HSC-like cells assessed by scRNAseq.

FIG. 9 is an example of data wherein glycolysis plays a role in hematopoietic specification. (a) Representative assay data shows the extracellular acidification rate (ECAR) measured in HE and EHT cells under basal conditions as well as after the addition of the indicated compounds. Bar graphs are shown in FIG. 2a. (b) Dot plots showing gene expression levels of glycolytic enzymes detected in human CS 13 AGM region (data from Zeng et al.), by mapping of their scRNAseq data onto our dataset (as shown in FIG. 1c) and based on percent expressed (size of the dots) and average level of expression (color intensity). (c) Glucose is broken down through glycolysis and the resulting pyruvate gives rise to lactate or converts to acetyl-coA for integration into the TCA cycle. 2-Deoxy-D-glucose (2-DG) blocks the glycolytic flux. (d) Subculture day 3 CD43+ cell frequencies±s.e.m. relative to the control are shown (n=7, paired t-test). (e) Subculture day 3 CD43+GPA+ and subculture day 6 CD43+CD45+ cell frequencies±s.e.m. relative to the control are presented (n=6 and n=5, respectively, paired t-tests). (f) Subculture day 3 CellTrace Violet (CTV) fluorescence was assessed by flow cytometry and median MFI values are shown (n=4, paired t-tests).

FIG. 10a is an example of data wherein OXPHOS is increased during EHT even in the absence of glucose. Representative assay data shows oxygen consumption rate (OCR) measured in HE and EHT cells under basal conditions as well as after the addition of the indicated compounds. Bar graphs are shown in FIG. 2, g.

FIG. 10b is an example of data wherein OXPHOS is increased during EHT even in the absence of glucose. Heatmap showing scRNAseq data of OXPHOS-related genes expressed in HE, EHT and HSC-like populations.

FIG. 10c is an example of data wherein OXPHOS is increased during EHT even in the absence of glucose. Heatmap showing scRNAseq data of OXPHOS-related genes expressed in human CS 13 AGM region (data from Zeng et al.), by mapping of their scRNAseq data onto our dataset (as shown in FIG. 1c). Note that more OXPHOS-related genes are detectable in this primary cell dataset.

FIG. 10d is an example of data wherein OXPHOS is increased during EHT even in the absence of glucose. Dotplot showing scRNAseq data of TCA cycle enzymes expressed in human CS 13 AGM region (data from Zeng et al.), by mapping of their scRNAseq data onto our dataset (as shown in FIG. 1c).

FIG. 10e is an example of data wherein OXPHOS is increased during EHT even in the absence of glucose. OCR was measured in HE and EHT cells (n=11) in the absence of glucose as well as after the addition of the indicated compounds. Corresponding bar graphs show mean levels±s.e.m. of OCR in the absence of glucose and after glucose injection (n=11, from 3 independent experiments, unpaired t-tests).

FIG. 11 is an example of data wherein glutamine contributes to distinct processes for inducing early erythroid and mature hematopoietic lineages. (a) Schematic showing the contribution of glutamine to the TCA cycle. Glutamine is deamidated to glutamate (Glu) which is then converted to α-ketoglutarate (α-KG), an intermediate of the TCA cycle. The conversion of glutamine to glutamate is mediated by the glutaminase (GLS) enzyme, which is specifically inhibited by BPTES. (b) Dot plots showing gene expression levels of glutamine transporters detected by scRNAseq and based on percent expressed (size of the dots) and average level of expression (color intensity). (c and d) FACS-sorted HE cells were subcultured with or without BPTES (25 μM). Subculture day 3 representative plots and bar graphs (c) show CD43+GPA+ cell frequency±s.e.m. (n=6, paired t-tests). Subculture day 6 representative plots and bar graphs (d) show CD43+CD45+ cell frequency±s.e.m. (n=4, paired t-tests). (e and f) FACS-sorted HE cells were subcultured in glutamine-free medium with the indicated compounds. Subculture day 3 representative plots for FSC-A/CD43 (e) are shown. Bar graphs of percentages of cells expressing CD43±s.e.m. at day 6 of subculture are depicted (f) (n=3, paired t-tests). (g) Bar graphs for the percentages of CD34−CD43+ cells±s.e.m. in FIG. 3, a are shown (n=3, paired t-tests). (h) CTV median MFIs±s.e.m. relative to control are shown (n=5, paired t-tests, see corresponding plot in FIG. 3, b). (i) CTV median MFIs±s.e.m. corresponding to FIG. 3, c are shown (n=6, paired t-tests). (j) FACS-sorted HE cells were subcultured in glutamine-free medium with the indicated compounds. Subculture day 3 or day 6 representative plots are shown for FSC-A/GPA and FSC-A/CD45 (see FIG. 3, d for graph). (e) Plots showing percentages of CD45+CD56+ cells obtained following 35-day co-culture of 3-day subcultured HE cells with OP9-DL1 stroma. During the 3-day subculture, HE cells were treated with the indicated compounds. ns, not significant, *p<0.05, **p<0.01, ***p<0.001.

FIG. 12 is an example of data wherein pyruvate catabolism directs hematopoietic lineage specification. (a) FACS-sorted HE, EHT or HSC-like cells were subcultured with or without UK5099 (10 μM). Subculture day 3 CD43+/GPA+ cell frequencies±s.e.m. relative to the controls for all populations are presented (HE, n=5; EHT, n=6; HSC-like, n=4; paired t-tests). (b) FACS-sorted HE cells were subcultured with or without 1-AA (4 mM). Subculture day 3 CD43+GPA+ cell frequencies±s.e.m. relative to the control are shown (n=4, paired t-test). (c) Fold change of expression of MPC1 and MPC2 relative to HPRT in shRNA-transduced cells compared to shScrambled (shScr) are shown (n=3, unpaired t tests). Untr, untransduced. (d) HE cells were transduced with shScrambled (shScr), shMPC1, shMPC2 or both the day after the sort and day 3 CD43+/GPA+ cell frequencies±s.e.m. relative to shScr are presented (n=4; one-way ANOVA test). Untr, untransduced. (e) FACS-sorted HE cells were stained with CTV and fluorescence was assessed by flow cytometry for GPA+ cells at day 3 of subculture with or without UK5099 (10 μM). Representative of n=3. (f-g) FACS-sorted HE, EHT or HSC-like cells were subcultured with or without UK5099 (10 μM). Subculture day 6 CD43+ (f) and CD43+CD45+ (g) cell frequencies±s.e.m. relative to the control for all populations are presented (HE, n=7; EHT, n=7; HSC-like, n=4; paired t-tests). (h) FACS-sorted HE cells were subcultured with or without 1-AA (4 mM). Subculture day 6 CD43+ and CD43+CD45+ cell frequencies±s.e.m. relative to the control are shown (n=3, paired t-test). (i-j) FACS-sorted HE cells were subcultured for 3 days with or without UK5099 (10 μM). CTV for HE-derived CD45+ cells (i) and HE-derived HSC-like cell frequencies±s.e.m. relative to the control (n=7, paired t-test) (j) are shown. (k-p) FACS-sorted HE and EHT cells were subcultured with or without DCA (3 mM). Subculture day 3 (k, n=3) and day 6 (l, HE, n=5; EHT, n=4) CD43+GPA+ cell frequencies±s.e.m. relative to the controls are shown (paired t-test). (m) CTV for HE-derived GPA+ cells at subculture day 3 is shown. Representative of n=3. (n) Subculture day 6 CD43+CD45+ cell frequencies±s.e.m. relative to the controls for both populations are shown (HE, n=5; EHT, n=4; paired t-tests). (o) CTV for HE-derived CD45+ cells are shown. Representative of n=3. (p) HE-derived HSC-like cell frequencies±s.e.m. relative to the control (n=4, paired t-test) are shown. (q) EdU incorporation into HE cells was assessed by flow cytometry after a 24 h pulse at days 1 and 2 of subculture with or without UK5099 (10 μM) or DCA (3 mM) (n=3). (r-s) Percentages of CFU assay colony types obtained from HE cells subcultured with the indicated compounds for 3 days (r) (n=3, 2-way ANOVA test) or 6 days (s) (n=5, 2-way ANOVA test). CFU, colony forming unit; E, erythroid; M, macrophage; G, granulocyte; GEMNI, mixed. (t) EryD and EryP CFU-Es obtained from HE cells subcultured with the indicated compounds for 3 days. Scale bars, 100 μm. (u) Fold change in the expression of HBA1-2 transcripts normalized to KLF1 in CFUs obtained from HE cells treated with UK5099 (10 μM) or DCA (3 mM) relative to non-treated cells. (v) Plots showing percentages of CD45+CD56+ cells obtained following 35-day co-culture of 3-day subcultured HE cells with OP9-DL1 stroma. During the 3-day subculture, HE cells were treated with the indicated compounds. ns, not significant, *p<0.05, **p<0.01, ***p<0.001.

FIG. 13 is an example of data wherein modulation of pyruvate metabolism affects lineage specification in vivo. (a-c) Pregnant mice were injected with UK5099 or DCA at E9.5 and fetal livers were analyzed at E14.5 by flow cytometry. FL, fetal liver. Levels of HPC-1, HPC-2 (a) and erythroid progenitors (b) as percentages in fetal liver are shown for control (n=10), UK5099-treated (n=14) and DCA-treated (n=16) conditions (one-way ANOVA test). (c) Erythroid differentiation stages according to CD71/Ter119 staining are shown on the control plot. Representative plots showing percentages of cells in each stage are shown for control, UK5099- or DCA-treated conditions. (d) Gating strategy is depicted for sorting LT-HSCs in E14.5 embryos. (e) Percentages of colonies obtained from sorted LT-HSCs are shown for control (n=4), UK5099-treated (n=8) and DCA-treated (n=10) conditions (one-way ANOVA test). (f-i) Irradiated NSG mice were transplanted with DCA-treated HE cells kept in co-culture with OP9-DL1 stroma for 3 days and human cells in peripheral blood (PB) were assessed on weeks 4, 8 and 12. (f) Engraftment levels in peripheral blood (PB) as percentages of huCD45+ cells are shown (Control, n=6; DCA, n=7). (g) Thymi were harvested on week 12 after transplantation and representative plots showing CD4/CD8 expressing cells are presented for control and DCA-treated conditions. (h) Representative plots and percentages±s.e.m. of CD19+ cells (B cells) in huCD45+ cells from PB at week 8 are shown (Control, n=6; DCA, n=6; unpaired t test). (i) Percentages±s.e.m. of human HSCs in huCD45+ cells from the BM are shown (Control, n=6; DCA, n=7; unpaired t tests). ns, not significant, *p<0.05, **p<0.01, ***p<0.001, ****p<0.0001.

FIG. 14 is an example of data showing expression of endothelial and hematopoietic genes in differentiating HE cells Single-cell RNAseq was performed on control, UK5099-treated and DCA-treated HE cells at day 2 of subculture. Feature plots showing the expression of endothelial (a) or hematopoietic (b) genes on the UMAP in FIG. 5, a. (c) 10×10 dot plots showing the percentages of cells belonging to clusters 6 and 7 in each condition. (d) Numbers of GPA+ clones obtained from single HE cells co-cultured on OP9-DL1 stroma, treated with the indicated compounds for 14 days (n=6 independent experiments, with a total of 552 wells screened for each condition).

FIG. 15 is an example of data showing mechanistic analyses of pyruvate catabolism during EHT. (a) FACS-sorted HE cells were subcultured with or without TSA (60 nM). Subculture day 3 CD43 MFI levels and a representative CD43 histogram are shown (n=4, paired t test). (b) Dot plots showing gene expression levels of LSD1, GFI1 and GFI1B detected by scRNAseq and based on percent expressed (size of the dots) and average level of expression (color intensity). (c) Fold change of expression of LSD1 relative to HPRT in shRNA-transduced cells compared to shScrambled (shScr) are shown (n=3, unpaired t tests). Untr, untransduced. (d-e) FACS-sorted HE cells were subcultured with TCP (300 nM), DCA (3 mM) or both. Day 6 CD43+CD45+ cell frequencies±s.e.m. (d) and Day 6 CD43+CD45+CD33+CD11b+ cell frequencies±s.e.m. (e) relative to the control are shown (n=5, oneway ANOVA test). (f) Acetate can be directly converted to acetyl-coA by ACSS2 (inhibitor: ACSS2i). Acetyl-coA is the precursor of acetylation marks, transferred onto histones via histone acetyltransferases (HATs, inhibitor: C646). (g-h) FACS-sorted HE cells were subcultured with ACSS2i (5 μM), DCA (3 mM), or both (n=5, one-way ANOVA test) (g) or with C646 (10 μM), DCA (3 mM), or both (n=3, oneway ANOVA test) (h) and day 6 CD43+CD45+ cell frequencies±s.e.m. are shown. (i) FACS-sorted HE cells were subcultured with or without DCA (3 mM) for 2 days on coverslips. Staining intensities of H3K9 acetylation and H4K5, 8, 12, 16 acetylation were assessed by confocal microscopy imaging and fold change compared to the control is shown (n=3). (j) Dot plots showing gene expression levels of cholesterol efflux pathway genes detected by scRNAseq and based on percent expressed (size of the dots) and average level of expression (color intensity).

### DETAILED DESCRIPTION

During embryonic development, hematopoiesis initially occurs through primitive and definitive waves primarily in the yolk sac (YS) and the aorta-gonad-mesonephros (AGM) regions, giving rise to distinct blood lineages (Palis, J. et al. Development 126, 5073-5084 (1999); and Medvinsky, A. & Dzierzak, E. Cell 86, 897-906 (1996)). The first hematopoietic stem cells (HSCs) emerge from hemogenic endothelial (HE) cells in the AGM, through endothelial to hematopoietic transition (EHT) (Boisset, J.-C. et al. Nature 464, 116-120 (2010); and Kissa, K. & Herbomel, P. Nature 464, 112-115 (2010)). In the adult, HSC quiescence, maintenance and differentiation are closely linked to changes in metabolism (Takubo, K. et al. Cell Stem Cell 12, 49-61 (2013); and Yu, W.-M. et al. Cell Stem Cell 12, 62-74 (2013)). In certain examples disclosed herein, de novo emergence of blood may be regulated by multiple metabolic pathways that directly induce or modulate hematopoietic specification and lineage commitment during human EHT. EHT may be accompanied by a metabolic switch, with concomitant increases in glycolysis and oxidative phosphorylation (OXPHOS). Moreover, the OXPHOS fuel glutamine may be essential for hematopoietic emergence and, through its different pathway intermediates, is able to direct distinct lineage outcomes. In both in vitro and in vivo settings, steering pyruvate use towards glycolysis or OXPHOS may differentially skews commitment of HE cells to either a primitive erythroid fate or a definitive fate with lymphoid/myeloid potential, respectively. In certain examples, the commitment to primitive or definitive fates in this context may be controlled by distinct mechanisms. During EHT, metabolism may be a major determinant of hematopoietic specification, lineage commitment and primitive versus definitive fate decisions. The disclosure provided herein may provide a basis for using modulation of metabolic pathways to generate definitive HSCs in vitro, in examples thereby providing an invaluable source of treatment for hematological disorders and malignancies.

Induced pluripotent stem (iPS) cells, because of their functional equivalence to embryonic stem cells may have unlimited self-renewal potential, and because they can be generated from somatic cells of the patient him/herself (e.g., skin cells, or amniotic fluid MSCs etc.) and thus recognized as self, is one such ideal source and perhaps the most feasible. In some examples, the ability to generate hematopoietic stem cells from patient derived iPS cells enables the generation of an unlimited supply of human leukocyte antigen (HLA) matched cells, capable of reconstituting the hematopoietic system of patients with hematological disorders or patients undergoing chemotherapy for hematopoietic and some non-hematopoietic solid tumor malignancies. In some examples, depending on the source of somatic cells for deriving the iPS cells, iPS derived hematopoietic stem cells may be superior to traditionally harvested hematopoietic stem cells in terms of: 1) reduced acquired mutations (e.g., if iPS cells were derived from neonatal cell sources), 2) unlimited expansion ability, 3) reduced rejection issues, 4) no contaminating cells from the original tumor present, and 5) the ability to correct congenital mutations in iPS cell lines from patients using existing gene editing technologies such as Crispr/Cas.

Moreover, recent advances in the ability to generate T-cells specifically designed to target and destroy malignant cells following their differentiation from iPS cells means that transplantations may be performed with simultaneous administration of stem cells and anti-tumor T-cells (Trounson et al. Nature Reviews 2016, Vizcardo et al. Cell Stem Cell 2013). As such, in some examples, the ability to generate iPS derived hematopoietic stem cells provides an immediate demand for donor cells for many patients, and potentially offers an exponential increase in use as the surrounding technologies advance. Thus, iPS derived hematopoietic cells offer a reliable and robust new treatment modality for patients with the aforementioned life-threatening diseases.

Further, the ability to generate therapeutically valuable mature or differentiated hematopoietic cells from iPS for transfusion into patients is another facet that perhaps is even greater in terms of serving a public need. In some examples, functional red cells can be generated en mass for all blood groups to be able to address the shortages of transfusion products for patient who have suffered blood loss as a result of injury, who require transfusions during surgery, or suffer from various forms of anemia. In addition, other blood cells differentiated from the iPS may also be useful in the treatment of cancer, such NK or T-cells programmed with antitumor activities.

The tricarboxylic acid (TCA) cycle, also known as the Krebs or citric acid cycle, is the main source of energy for cells and an important part of aerobic respiration. The cycle harnesses the available chemical energy of acetyl coenzyme A (acetyl CoA) into the reducing power of nicotinamide adenine dinucleotide (NADH). The TCA cycle is part of the larger glucose metabolism whereby glucose is oxidized to form pyruvate, which is then oxidized and enters the TCA cycle as acetyl-CoA.

Differentiating iPS cells function like embryonic stem (ES) cells. Unlike ES cells, iPS cells are more readily obtainable for therapy and research, and their isolation does not carry the same ethical concerns. Human iPS cells may be an ideal source for patient-specific therapy since they can be derived from the patients themselves. In addition, iPS cells can serve as useful research tools by providing models of human disease to use for screening new drugs or for studying mechanisms of pathogenesis and toxicology, and models of normal development.

Hematopoietic stem cells (HSCs) are undifferentiated cells whose progeny reconstitute blood cells lineages such as monocytes/macrophages or T and B lymphocytes through a process called hematopoiesis. HSCs possess an indefinite self-renewal potential explaining the interest in these cells for transplantation for the sustained reconstitution of blood cells. B cells are a type of lymphocytes responsible for the humoral immunity (immunity mediated by antibodies).

Definitive hematopoietic stem cells (HSCs) are responsible for the continuous production of all mature blood cells during the entire adult life span of an individual. They are clinically important cells in transplantation protocols used in therapies for blood-related diseases. Experimentally, HSCs can confer long-term reconstitution of the entire hematopoietic system of an irradiated adult recipient.

In certain examples, specific metabolic pathway regulators of glycolysis and the TCA cycle (principle means of energy production in the cell) may directly activate transcriptional changes in the precursors of hematopoietic cells (cells undergoing endothelial to hematopoietic transition) that allows for directed hematopoietic lineage biasing and generation of definitive hematopoietic cells. The ability to generate definitive hematopoietic cells from reprogrammed cells is critical for therapeutics because only definitive cells give rise to the lymphoid blood lineages, (NK, B-cells and T-cells), hematopoietic stem cells, and erythroid (red) cells that express adult hemoglobins. These are the cell types that are currently provided by donors that are already widely used or being developed for use hematopoietic cell-based therapies, including the millions of red cells transfusions that patients receive worldwide each year.

In addition to the modulation of the metabolic pathways described above in iPS derived production of definitive hematopoietic cells, metabolic modulation may be an important means for generating definitive blood from cells sources other than iPS cells. For example, de novo generation of definitive hematopoietic cells may be achieved by direct reprogramming of somatic cells into precursor cells of blood including mesodermal cells and cells undergoing endothelial to hematopoietic transition, also in addition to directly reprogrammed blood cells. Metabolic modulation may provide a basis to guide definitive blood production in all these cases. Moreover, the ability for self-renewal of already committed definitive blood cells (i.e., from bone marrow, cord blood, mobilized peripheral blood, which are currently used in hematopoietic stem cell transplantation therapies worldwide today) benefits from metabolic pathway manipulation for the self-renewal and expansion of the therapeutic hematopoietic stem cell or other definitive hematopoietic cells.

Pyruvate dehydrogenase kinase family members (PDK1, PDK2, PDK3, PDK4) are serine kinases that catalyze phosphorylation of the E1α subunit of the pyruvate dehydrogenase complex (PDC). Pyruvate dehydrogenase kinase is activated by ATP, NADH and acetyl-CoA. It is inhibited by ADP, NAD+, CoA-SH and pyruvate. Biochemicals that inhibit PDK may be used to direct hematopoietic lineage biasing and to generate definitive hematopoietic cells. For example, inhibitors of Pyruvate dehydrogenase kinases (PDK) include Leelamine HCl, a weak CB1 receptor agonist and PDK inhibitor; Quercetin Dihydrate, a natural flavonoid antiproliferative kinase inhibitor; Sodium dichloroacetate, an inhibitor of mitochondrial pyruvate dehydrogenase kinase; SB 203580 (hydrochloride), a MAPK inhibitor; Dichloroacetic acid, a mitochondrial PDK (pyruvate dehydrogenase kinase) inhibitor; PDK1/Akt/Flt Dual Pathway Inhibitor, which is a cell-permeable compound that selectively induces apoptosis; BX 795, an inhibitor of PDK1, TBK1, and IKK&epsilon; SB 203580; a pyridinyl imidazole and specific inhibitor that suppresses p38 mediated activation of MK2; KT 5720, a potent, specific and cell-permeable inhibitor of PKA; BX-912, a potent and selective PDK-1 inhibitor that induces apoptosis; GSK 2334470, a potent and selective PDK1 inhibitor that subsequently induces apoptotic cell death; and OSU 03012, a PDK1 inhibitor and inducer of caspase and p53-independent apoptosis.

Pyruvate dehydrogenase (PDH) is the first component enzyme of pyruvate dehydrogenase complex (PDC). The pyruvate dehydrogenase complex contributes to transforming pyruvate into acetyl-CoA by a process called pyruvate decarboxylation (Swanson Conversion). Acetyl-CoA may then be used in the citric acid cycle to carry out cellular respiration. Thus, pyruvate dehydrogenase links the glycolysis metabolic pathway to the citric acid cycle and releasing energy via NADH. Pyruvate dehydrogenase may be allosterically activated by fructose-1,6-bisphosphate and is inhibited by NADH and acetyl-CoA. Phosphorylation of PDH is mediated by pyruvate dehydrogenase kinase. Metabolic regulators may be used that activate Pyruvate Dehydrogenase complexes (PDH).

The PDH inhibitor 1-aminoethylphosphinic acid (1-AA) may be used in a culture media for source cells, wherein the concentration of the 1-AA is preferably at least 4 μM, but may be in the range of about 0.5 μm to 50 μm, for example, about: 0.5 μm, 0.6 μm, 0.7 μm, 0.8 μm, 0.9 μm, 1.0 μm, 5 μm, 10 μm, 20 μm, 30 μm, 40 μm and 50 μm.

In some examples, metabolic regulators may be used to increase uptake of pyruvate into mitochondria. Transport of pyruvate across the outer mitochondrial membrane (OMM) is accomplished via large non-selective channels, such as voltage-dependent anion channels/porin, which enable passive diffusion (Benz R. Biochim Biophys Acta. 1994; 1197: 167-196). Voltage-Dependent Anion Channel (VDAC) is the most abundant protein in the OMM and serves as the main pathway for metabolite/ion transport between the cytosol and the intermembrane space (IMS) of mitochondria. Deficiencies in these channels have been suggested to block pyruvate metabolism (Huizing M. et al. Pediatr Res. 1996; 39: 760-765). Inhibitors of voltage-dependent anion channels/porin may be used to inhibit uptake of pyruvate. VDAC phosphorylation by protein kinases, GSK3β, PKA, and protein kinase C epsilon (PKCε), blocks or inhibits association of VDAC with other proteins, such as Bax and tBid, and also regulates VDAC opening. PKA-dependent VDAC phosphorylation and GSK3β-mediated VDAC2 phosphorylation increase VDAC conductance.

The movement of metabolites, such as pyruvate, through the inner mitochondrial membrane (IMM) may be more restrictive than across the OMM, however. Many metabolites have specific mitochondrial inner membrane transporters that have been identified and studied (Palmieri F. et al. Biochim Biophys Acta. 1996; 1275: 127-132).

Metabolic regulators may be used that accelerate conversion of pyruvate to acetyl coenzyme A (Ac-CoA). Dichloroacetate (DCA) promotes pyruvate entry into the Krebs cycle by inhibiting pyruvate dehydrogenase (PDH) kinase and thereby maintaining PDH in the active dephosphorylated state. In instances where the metabolic regulator is dichloroacetate (DCA), the concentration of dichloroacetate in a culture media for the source cells may be at least about 30 μM and can vary from 10 μM to 100 μM, including concentrations of about: 10 μM, 20 μM, 30 μM, 40 μM, 50 μM, 60 μM, 70 μM, 80 μM, 90 μM and 100 μM.

A metabolic regulator used in the disclosed methods herein may inhibit conversion of pyruvate to Ac-CoA. For example, UK-5099 is a potent inhibitor of the mitochondrial pyruvate carrier (MPC). UK-5099 inhibits pyruvate-dependent O₂ consumption with an IC₅₀ of 50 nM. The concentration of UK5099 in a culture media for the source cells may be at least 100 nM, but may be in the range of from 10 nM to 1 μm, including about: 10 nM, 20, nM, 30 nM, 40, nM, 50 nM, 60, nM, 70 nM, 80 nM, 90 nM, 100 nM, 0.1 μm, 0.2 μm, 0.3 μm, 0.4 μm, 0.5 μm, 0.6 μm, 0.7 μm, 0.8 μm, 0.9 μm and 1 μm.

Lysine-Specific Demethylase 1 (LSD1) may be used for EHT and particularly the erythroid lineage. Numerous LSD1 inhibitors have been reported such as TCP, ORY-1001, GSK-2879552, IMG-7289, INCB059872, CC-90011, ORY-2001 and RO7051790. One or more of these inhibitors may be used in combination, such as two or more, three or more, four or more, five or more, or combinations of six or more.

Metabolic regulators may be used that increase production of α-ketoglutarate. For example, L-glutamine is a nutritionally semi-essential amino acid for proper growth in most cells and tissues, and plays an important role in the determination and guarding of the normal metabolic processes of the cells. With the help of transport systems, extracellular L-glutamine may cross the plasma membrane and be converted into alpha-ketoglutarate (AKG) through two pathways, namely, the glutaminase (GLS) I and II pathways. Different steps of glutamine metabolism (the glutamine-AKG axis) may be regulated by several factors (Xiao, D. et al. 2016 Amino Acids 48: 2067-2080), rendering the glutamine-AKG axis a potential target to regulate generation of definitive hematopoietic cells from source cells by activation of a tricarboxylic acid cycle of the source cells. α-Ketoglutarate is membrane-impermeable, meaning that it is usually added to cells in the form of esters such as dimethyl α-ketoglutarate (DMKG), trifluoromethylbenzyl α-ketoglutarate (TFMKG) and octyl α-ketoglutarate (O-KG). Once these compounds cross the plasma membrane, they may be hydrolyzed by esterases to generate α-ketoglutarate, which remains trapped within cells. All three compounds increase intracellular levels of α-ketoglutarate. Thus, these compounds are metabolic regulators of α-ketoglutarate. In some examples, the concentration of dimethyl α-ketoglutarate in a culture media for the differentiating iPS cells may be at least about 17.5 μM, but may be from about 10 μM to 100 μM, including concentrations of 10 μM, 20 μM, 30 μM, 40 μM, 50 μM, 60 μM, 70 μM, 80 μM, 90 μM and 100 μM.

Various metabolic regulators disclosed herein may be used in combination with nucleosides, wherein the concentration of nucleosides may be at least 0.7 mg/L, but may be from about 0.1 mg/L to about 10 mg/L, including concentrations of about: 0.1 mg/L, 0.2 mg/L, 0.3 mg/L, 0.4 mg/L, 0.5 mg/L, 0.6 mg/L, 0.7 mg/L, 0.8 mg/L, 0.9 mg/L, 1 mg/L, 1.5 mg/L, 2 mg/L, 2.5 mg/L, 1 mg/L, 1.5 mg/L, 2 mg/L, 2.5 mg/L, 1 mg/L, 1.5 mg/L, 2 mg/L, 2.5 mg/L, 3 mg/L, 3.5 mg/L, 4 mg/L, 4.5 mg/L, 5 mg/L, 5.5 mg/L, 6 mg/L and 6.5 mg/L, 7 mg/L, 7.5 mg/L, 8 mg/L, 8.5 mg/L, 9 mg/L, 9.5 mg/L, 10 mg/L and 10.5 mg/L. The nucleosides comprise at least one of Cytidine, Guanosine, Uridine, Adenosine and Thymidine, but may include any potential combination such as two, three, four, or all five nucelosides.

### EXAMPLE 1

### Recapitulation of Human EHT and Hematopoietic Differentiation In Vitro

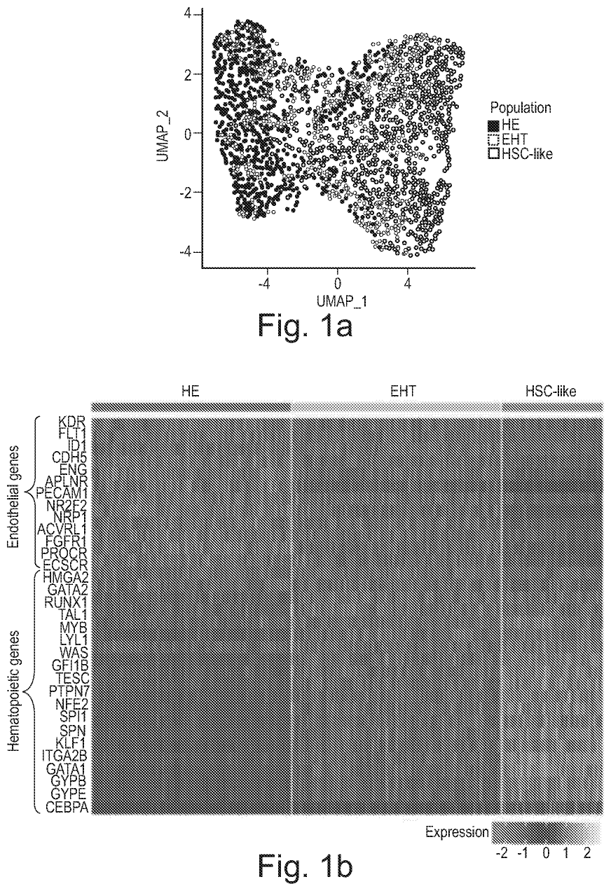

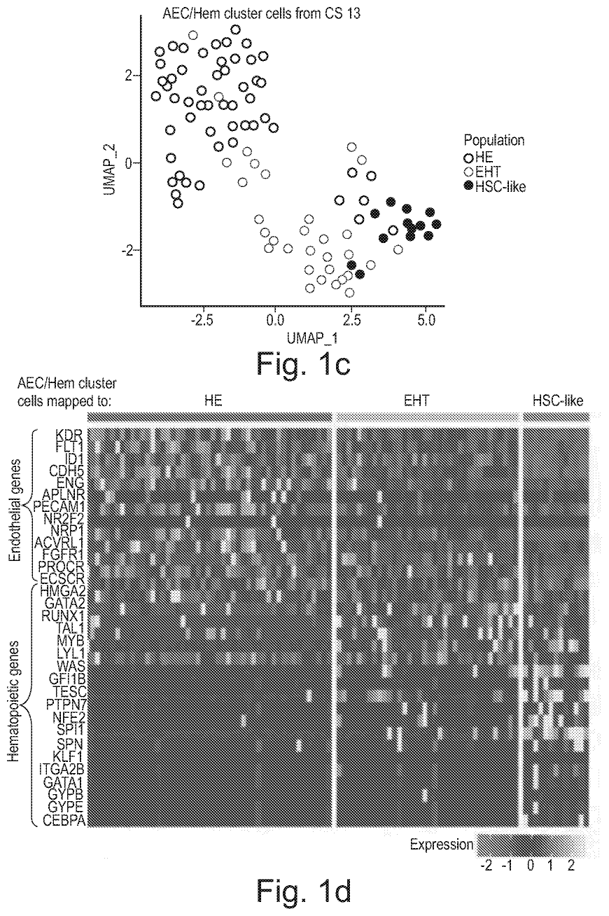

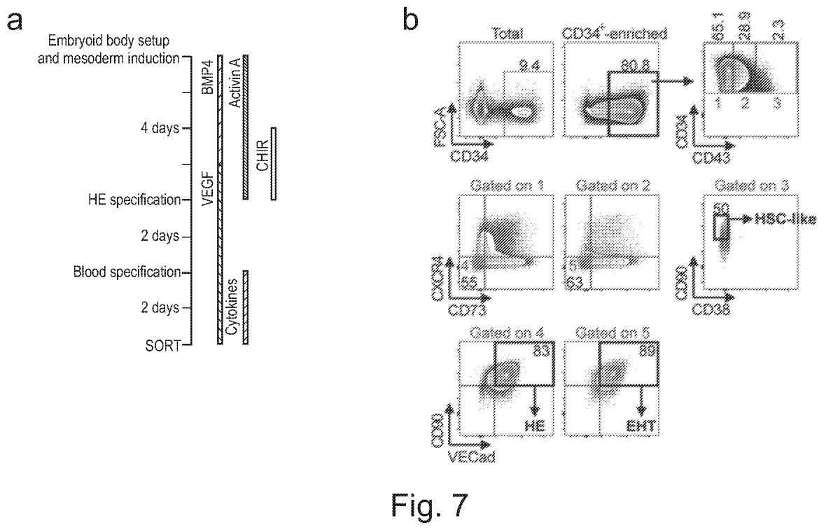

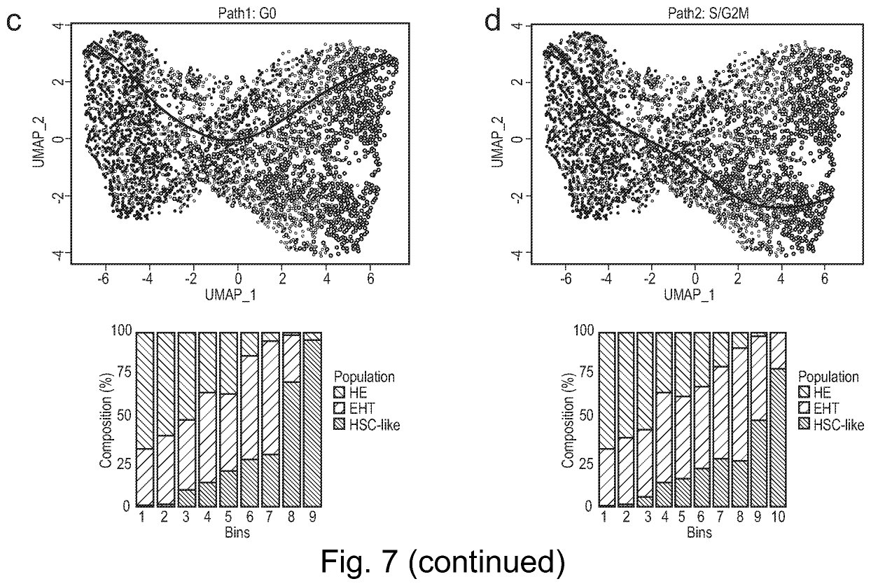

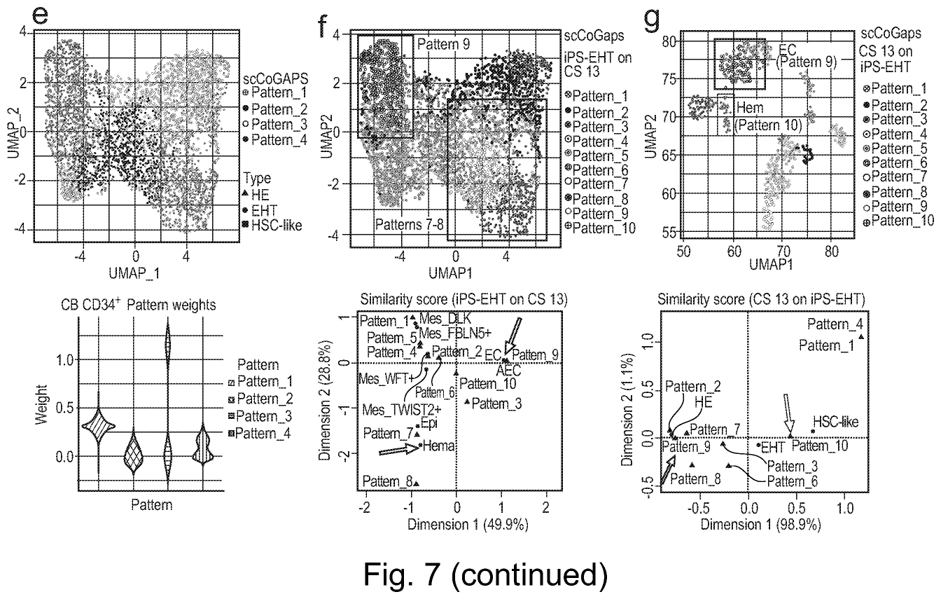

In an example to obtain both primitive and definitive hematopoietic waves in culture, two previously described small molecules were combined during human iPSC differentiation (FIG. 7, a): CHIR99021, a WNT pathway agonist which supports definitive hematopoiesis (Ng, E. S. et al. Nature Biotechnology 34, 1168-1179 (2016)) and Activin A, which promotes primitive hematopoiesis (Kennedy, M. et al. Cell Reports 2, 1722-1735 (2012)). After integrating these modifications to a previously described hematopoietic differentiation protocol (Ditadi, A. & Sturgeon, C. M. Methods 101, 65-72 (2016)), hemogenic endothelial cells (HE) were obtained, transitioning cells which express CD43 at intermediate levels (EHT) (Guibentif, C. et al. Cell Reports 19, 10-19 (2017)) and hematopoietic stem-like cells (HSC-like, immunophenotypical HSCs) (for gating strategies, see FIG. 7. b). These three populations were characterized transcriptionally using single-cell RNA sequencing (scRNAseq). The UMAP visualization placed the HE cells distally from HSC-like cells, with EHT cells bridging these two populations, confirming the sequential EHT process (FIG. 1a). Additionally, pseudotime analyses of the dataset was performed, taking two cell cycle paths, G0 (FIG. 7, c) and S/G2M (FIG. 7, d). In both instances, an abundance of HE cells was observed at the start, EHT cells in the middle and HSC-like cells at the end of the trajectory (FIG. 7, c and d; bar graphs). HE cells expressed endothelial markers such as KDR, FLT1, CDH5 but no hematopoietic markers; in contrast, EHT cells expressed both endothelial and hematopoietic markers and HSC-like cells only expressed hematopoietic markers such as RUNX1, TAL1, WAS and SPN (FIG. 1b), as shown previously in other EHT systems (Zhou, F. et al. Nature 533, 487-492 (2016); Swiers, G. et al. Nat Commun 4, 2924 (2013); and Guibentif, C. et al. Cell Reports 19, 10-19 (2017)). An isolated cord blood CD34⁺ cells dataset was generated and projected onto an EHT process dataset using the scCoGAPS package and observed that the highest pattern weights were for pattern 1 and part of pattern 3, both encompassing our HSC-like cluster (FIG. 7, e), proving that cord blood CD34⁺ cells share the most transcripts with the iPSC-derived HSC-like cells described elsewhere herein. The EHT process data was compared to a recently published scRNAseq analysis of primary human embryonic cells at Carnegie stage (CS) 13 (Zeng, Y. et al. Cell Res 1-14 (2019)). Of the 99 cells in the arterial endothelial and hematopoietic (AEC/Hem) clusters, 50, 36 and 13 cells mapped to the HE, EHT and HSC-like populations, respectively (FIG. 1c); and they clustered similarly to the EHT dataset in FIG. 1a. Furthermore, similarly to the EHT dataset, AEC/Hem cluster cells mapped to HE expressed endothelial markers such as KDR, FLT1, CDH5 and cells mapped to HSC-like cells expressed hematopoietic markers like RUNX1, TAL1, WAS and SPN (FIG. 1d). The dataset was mapped onto the human CS 13 dorsal aorta population dataset from Zeng et al. using the scCoGAPS package. A major part of the HE population (Pattern 9) colocalized with the CS 13 AEC and EC populations (grey arrow), while a significant part of the HSC-like population (Patterns 7-8) mapped to the CS 13 Hem cluster (pink arrow) (FIG. 7, f). When reverse mapping of CS 13 data was performed onto the dataset, the CS 13 EC population mapped close to the HE cells (Pattern 9, grey arrow) and the CS 13 Hem cluster mapped close to both the EHT and HSC-like cells (Pattern 10, green arrow) (FIG. 7, g). Thus, in examples, the system successfully captured the human EHT process and the HE, EHT and HSC-like populations which are obtained possess hemato-endothelial transcriptional signatures equivalent to that of the cell types that occur in the human embryo at CS 13.

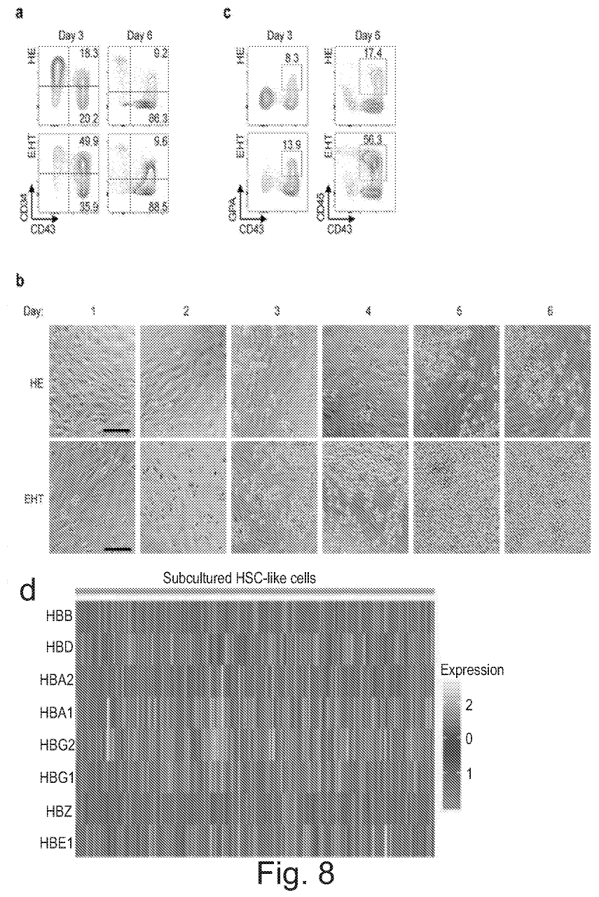

Next, the hematopoietic potential of both HE and EHT populations was verified. Both cell types gave rise to hematopoietic cells (CD43⁺) (FIG. 8, a). At day 6 of subculture, almost all cells (>96%) deriving from HE or EHT cells were CD43⁺ and the majority had lost CD34 expression (>86%), pointing to their maturation. In both cell cultures, spindle-shaped endothelial cells changed their morphology to round hematopoietic cells (FIG. 8, b). In both HE and EHT-derived subcultures, an erythroid (CD43⁺GPA⁺) cell population and a non-erythroid pan-hematopoietic CD43⁺CD45⁺ cell population were clearly discernible at days 3 and 6, respectively (FIG. 8, c). According to the model described by Kennedy et al. (Kennedy, M. et al. Cell Reports 2, 1722-1735 (2012)), the timeframes in which the CD43⁺GPA⁺ and the CD43⁺CD45⁺ cell populations are generated hints towards their primitive and definitive natures, respectively. Moreover, the presence of embryonic (HBZ, HBE1), fetal (HBA1, HBA2, HBG1, HBG2) and adult (HBD, HBB) globin upregulation in subcultured HSC-like cells further supports that, in this setting, we obtain both primitive and definitive hematopoietic cells (FIG. 8, d). Altogether, these results show that this differentiation system allows one to accurately model the human EHT process and subculturing the resulting HE cells efficiently gives rise to primitive and definitive hematopoietic populations.

### Glycolysis May Fuel Distinct Processes During EHT

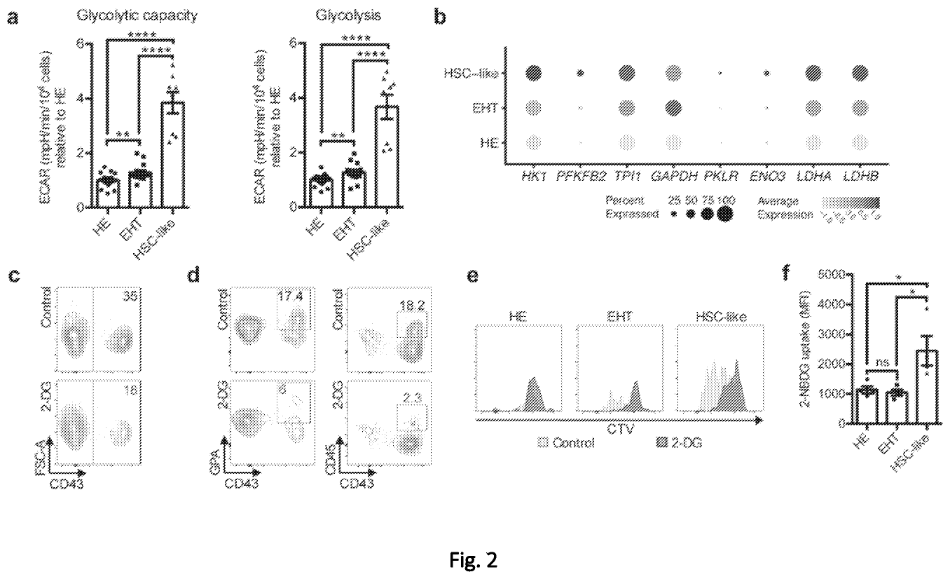

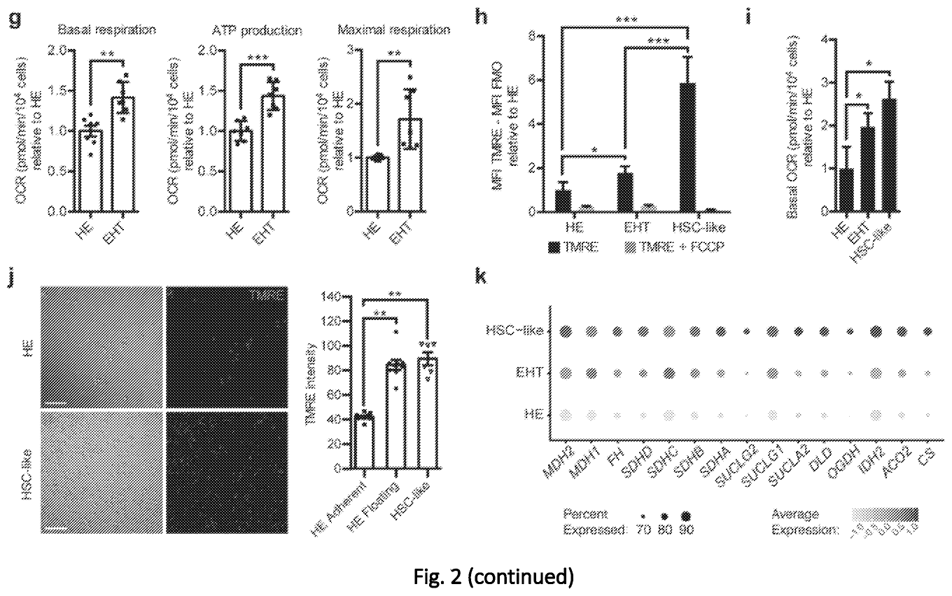

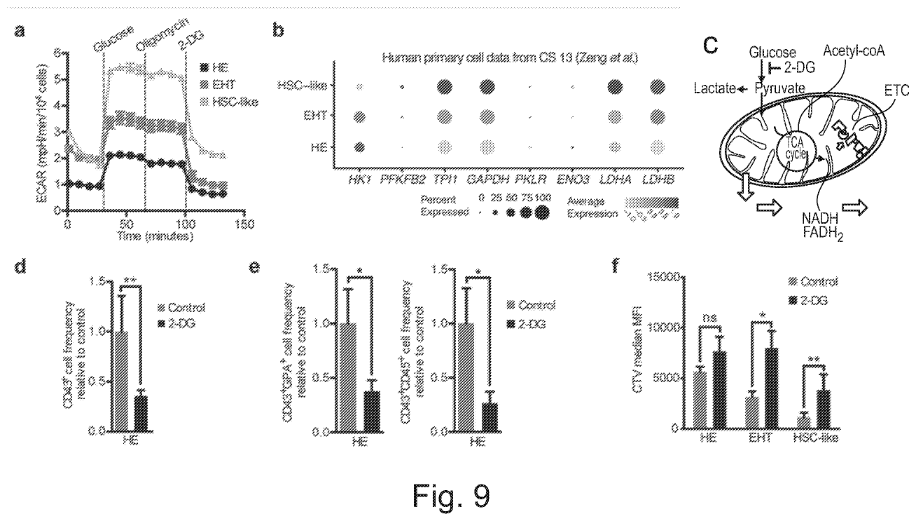

In examples, in order to describe the metabolic processes occurring in EHT populations, glycolysis was assessed in HE, EHT and HSC-like cells. A gradual increase in glycolytic capacity and glycolysis with differentiation was shown (FIG. 2, a, FIG. 9, a). Moreover, expression of the glycolytic enzymes HK1, PFKFB2, TPI1, GAPDH, PKLR, ENO3, LDHA and LDHB as assessed by scRNAseq also increased during EHT (FIG. 2, b). In some examples, an increase in the majority of these glycolytic enzymes during EHT in human primary cells from CS 13 was also shown (Zeng, Y. et al. Cell Res 1-14 (2019)), aligning with in vitro results (FIG. 9, b).

In an example exploring whether glycolytic activity is required during EHT, HE cells were treated with a glucose analog, 2-Deoxy-D-glucose (2-DG), which blocks glycolysis (FIG. 9, c). This treatment significantly reduced CD43⁺ cell output from HE cells at day 3 of subculture (FIG. 2, c, FIG. 9, d). Moreover, in the presence of 2-DG, generation of the CD43⁺GPA⁺ cell population at day 3 and the CD43⁺CD45⁺ cell population at day 6 was significantly impaired, dropping to less than 50% of the control (FIG. 2, d, FIG. 9, e). Intriguingly, proliferation rates of EHT or HSC-like cells, but not HE, were significantly reduced in the presence of 2-DG (FIG. 2, e, FIG. 9, f). These results suggest that while glycolysis is important for HE cells to induce hematopoietic differentiation, it may also fuels the proliferation of EHT and HSC-like cells at later steps during the EHT process.

### Mitochondrial Respiration May Gradually Increase During the EHT Process

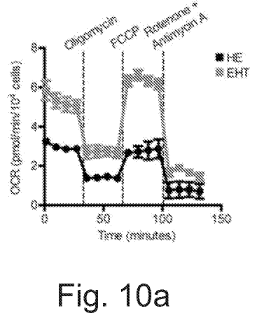

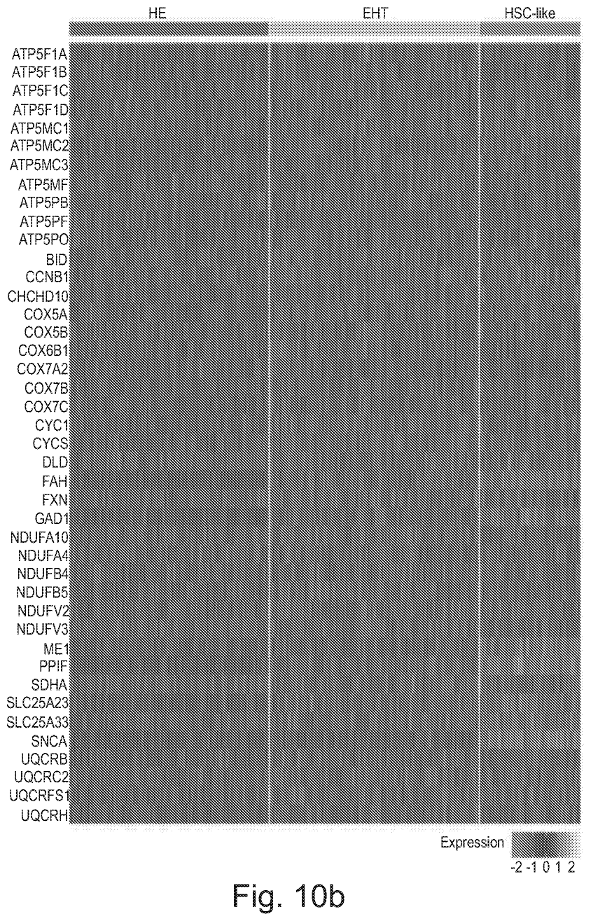

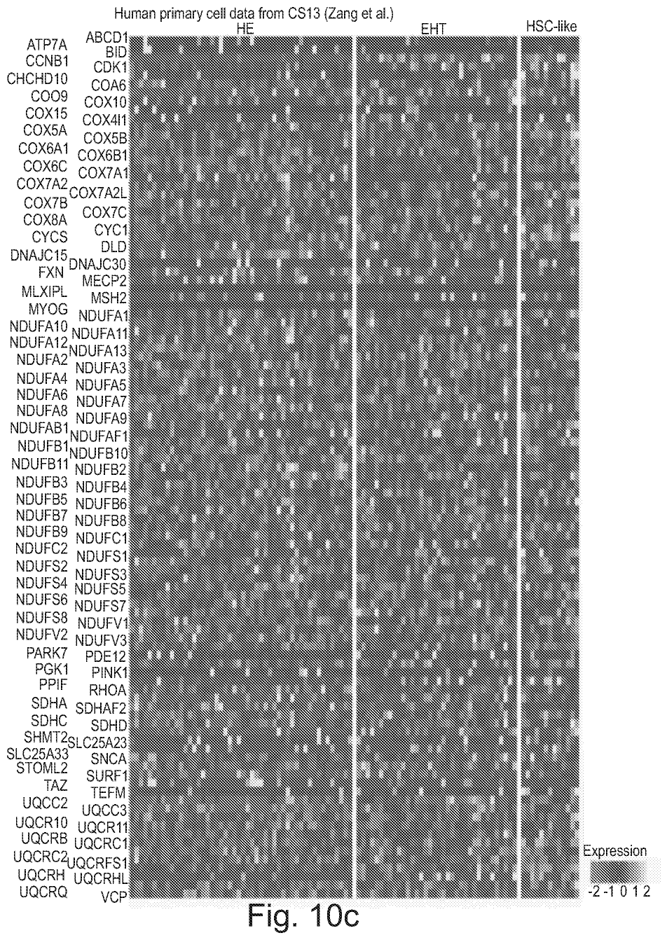

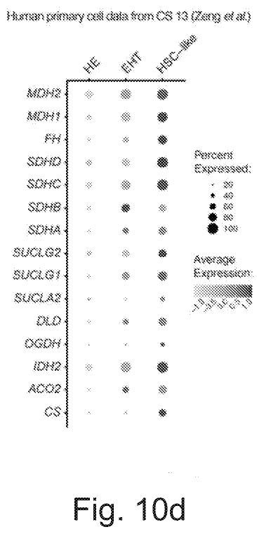

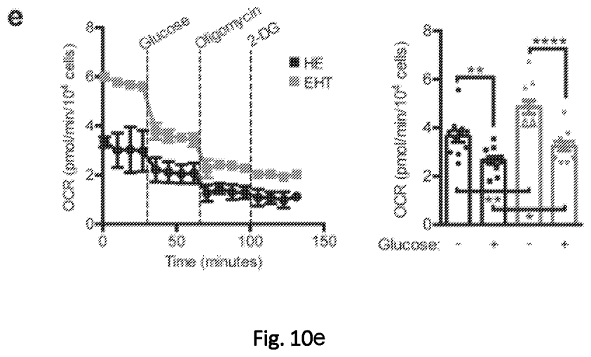

Along with increased glycolysis and proliferation, HSC-like cells also had increased glucose uptake compared to HE and EHT cells (FIG. 2, f). Interestingly, even though glycolytic flux was higher in EHT cells as compared to HE cells, glucose uptake was comparable in these two cell types. This result prompted us to investigate whether mitochondrial respiration was more active in HE versus EHT cells. Unexpectedly, EHT cells displayed higher levels of basal respiration, ATP production and maximal respiration as compared to HE cells (FIG. 2, g, FIG. 10, a). Moreover, mitochondrial activity measured by TMRE staining was significantly increased in individually analyzed EHT cells compared to HE cells and we observed an even higher rate in the case of HSC-like cells (FIG. 2, h). Treatment with FCCP, which depolarizes mitochondria, abrogated the TMRE signal in all cell types, suggesting that OXPHOS was active in these populations (FIG. 2, h). In line with the TMRE staining, the highest basal respiration rates we detected were in HSC-like cells (FIG. 2, i). Using live cell imaging by confocal microscopy, we compared mitochondrial activity in spindle-shaped HE cells versus their newly-formed round hematopoietic progeny in the same well. The TMRE staining intensity measurements showed a 2-fold higher mitochondrial activity in round as compared to spindle-shaped cells in HE wells and this value was similar to the levels detected in HSC-like cells (FIG. 2,j). Moreover, we observed a gradual increase in the expression of several genes implicated in OXPHOS, including subunits of Complex I (genes termed NDUF), II (SDHA), IV (genes termed COX) and V (genes termed ATP5) in HE, EHT and HSC-like populations by scRNAseq (FIG. 10, b). This result was accompanied by a progressive increase in TCA cycle enzymes during EHT (FIG. 2, k). We observed a gradual increase in both OXPHOS-related genes and TCA cycle enzymes during EHT in human primary cells at CS 13 (Zeng, Y. et al. Cell Res 1-14 (2019)), confirming our in vitro findings (FIG. 10, c and d). Taken together, these results show that TCA cycle activity, mitochondrial respiration and OXPHOS gradually increase during the EHT process.

### Glutamine May be the Limiting Step Initiating the Hematopoietic Differentiation of HE

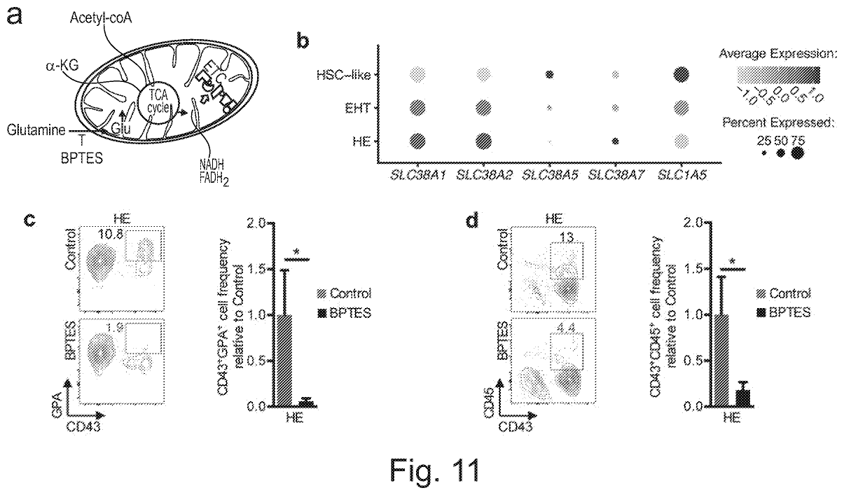

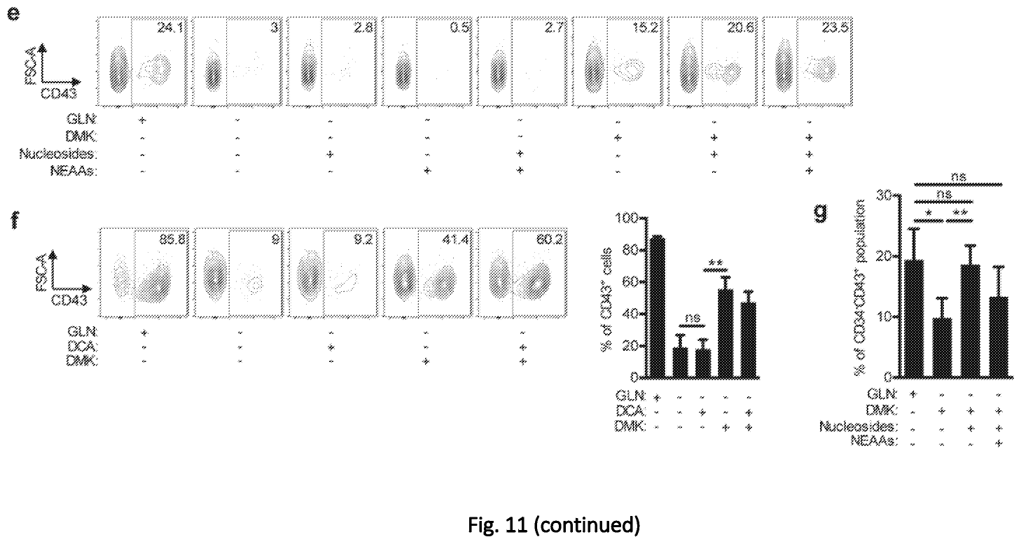

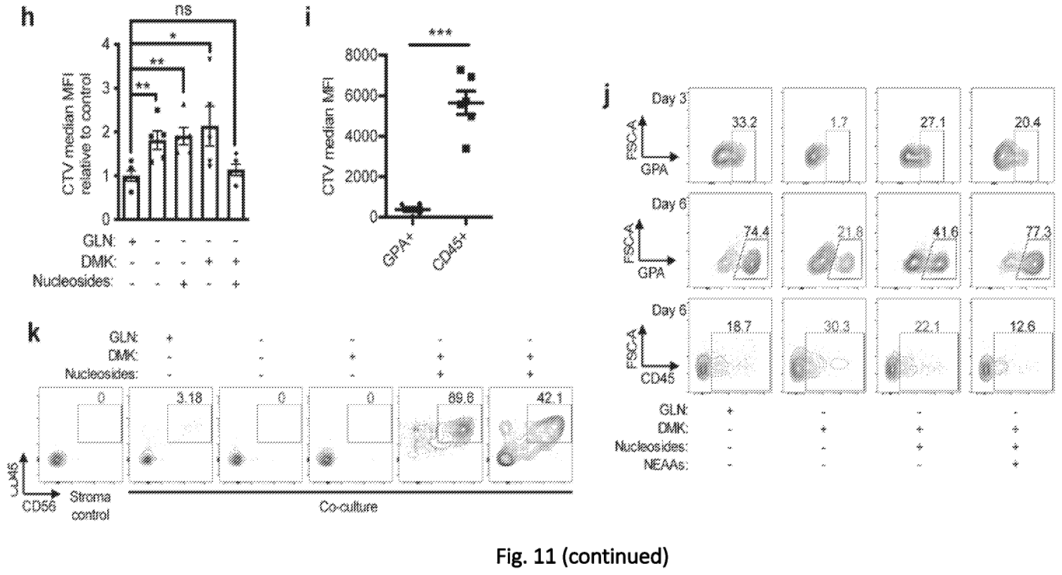

Even in glucose-free medium, HE and EHT cells had high basal respiration levels (FIG. 10, e). Thus, these cells may also rely on other energy sources for mitochondrial respiration. Glutamine can give rise to α-ketoglutarate (α-KG), an intermediate of the TCA cycle, and consequently feeds OXPHOS (FIG. 11, a). As shown in the figures, HE, EHT and HSC-like cells expressed several different glutamine transporters (FIG. 11, b) and HSC-like cells expressed the highest levels of the SLC1A5 transporter, as described previously in primary cord blood HSCs (Oburoglu, L. et al. Cell Stem Cell 15, 169-184 (2014)).

To determine whether glutamine is important for EHT, glutaminase (GLS) enzyme was blocked, which catalyzes the deamidation of glutamine to glutamate (FIG. 11, a), by treating HE cells with BPTES. A sharp decline in the generation of HE-derived CD43⁺GPA⁺ erythroid population at day 3 and CD43⁺CD45⁺ population at day 6 in the presence of BPTES is shown in the figures (FIG. 11, c and d). This result suggests that, in some examples, the entry of glutamine into the TCA cycle may be needed for hematopoietic differentiation during EHT.

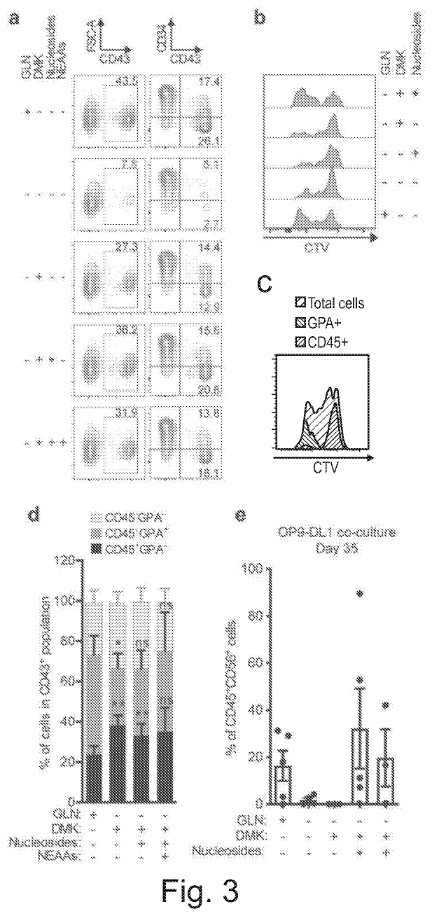

Glutamine also participates in several metabolic pathways including nucleotide and non-essential amino acid (NEAA) syntheses (DeBerardinis, R. J. & Cheng, T. Oncogene 29, 313-324 (2009)). Therefore, to get a better grasp of its role during EHT, HE cells in its absence. Glutamine deprivation abolished CD43⁺ cell output (>80% decrease) from HE cells at day 3 of subculture (FIG. 3, a). To rescue this phenotype, the glutamine-free culture medium was supplemented with nucleosides, NEAAs or a cell-permeable form of α-KG (dimethyl-ketoglutarate, DMK), all of which are substrates which can derive from glutamine (DeBerardinis, R. J. & Cheng, T. Oncogene 29, 313-324 (2009)). Nucleosides, NEAAs or a combination of both could not rescue the effect seen in glutamine deprivation (FIG. 11, e). However, DMK addition rescued CD43⁺ cell output from HE cells up to 60% (FIG. 3, a, FIG. 11, e). Moreover, a combination of DMK/nucleosides, or DMK/nucleosides/NEAAs further increased the percentage of CD43⁺ cells deriving from HE cells, reaching the levels in the control condition.

As pyruvate, another fuel for the TCA cycle, could replace glutamine, we treated HE cells with a pyruvate dehydrogenase kinase (PDK) inhibitor, dichloroacetate (DCA), to increase pyruvate dehydrogenase (PDH) activity during glutamine deprivation. Without glutamine, in examples, DCA treatment alone could not restore the CD43⁺ cell levels seen in the control (FIG. 11, f).

In certain examples, the percentage of more mature CD43⁺ cells that have lost CD34⁺ expression was significantly decreased in the glutamine-free DMK-treated condition as compared to the control (FIG. 3, a, FIG. 11, g). However, nucleoside addition alone or together with NEAAs restored the percentage of CD43⁺CD34⁻ cells to the levels observed in the control. As nucleotides are essential in proliferating cells, in certain examples, the proliferation of differentiating HE cells depended on this factor. In some examples, DMK or nucleosides alone could not restore the proliferation profile seen in the control condition; indeed, only the addition of both these factors reinstated the proliferation of HE cells during glutamine deprivation (FIG. 3, b, FIG. 11. h). These results indicate that glutamine may be important for the generation of CD43⁺ cells from the HE and that it plays a role in TCA cycle fueling as well as in nucleotide production for proliferative support.

### Glutamine Differentially Sustains Hematopoietic Populations

Previously, erythroid differentiation has been shown to require glutamine-fueled nucleotide synthesis (Oburoglu, L. et al. Cell Stem Cell 15, 169-184 (2014)). Thus, HE cells were stained with a proliferation dye (Cell Trace Violet, CTV) and the proliferation status of newly-formed GPA⁺ or CD45⁺ cells was assessed 3 days later. While GPA⁺ cells clustered to the divided cells (low CTV MFI values), interestingly, CD45⁺ cells deriving from HE cells had few to no divisions (high CTV MFI values; FIG. 3, c, FIG. 11, i). At days 3 and 6 of the HE subcultures without glutamine, DMK alone could not rescue the CD43⁺GPA⁺ population to the levels seen in the control (FIG. 11, j). However, a combination of DMK/nucleosides or DMK/nucleosides/NEAAs gave rise to a CD43⁺GPA⁺ population comparable to the one seen in the presence of glutamine (FIG. 3, d, FIG. 11, j). Consequently, glutamine acts as both a carbon- and nitrogen-donor to produce α-KG and nucleotides, which are both required for the production of CD43⁺GPA⁺ from HE cells, in line with their proliferation profile (FIG. 3, c).

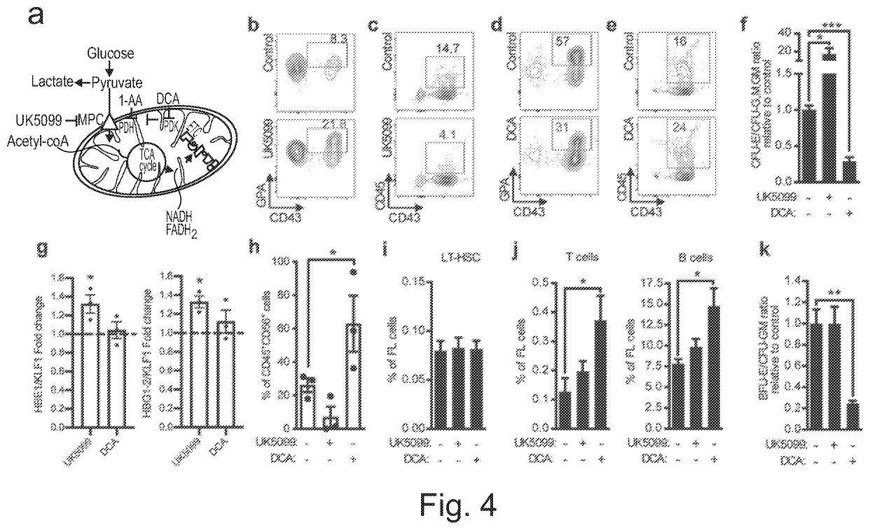

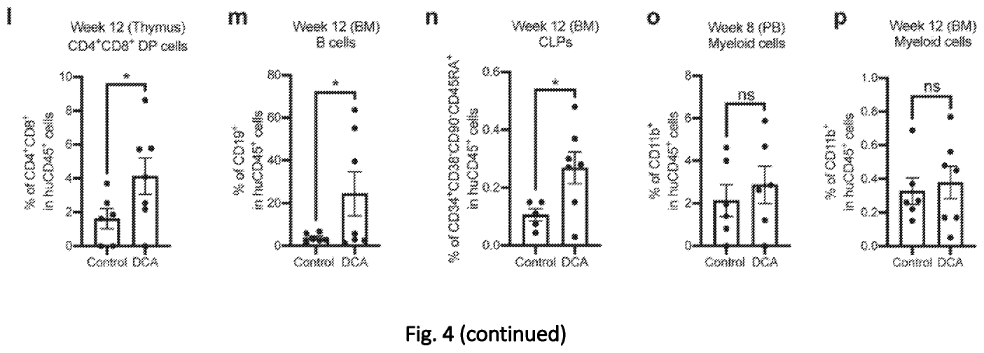

Interestingly, a significant increase in the percentages of CD43⁺CD45⁺ cells in conditions was restored by DMK or DMK/nucleosides (FIG. 3, d, FIG. 11, j) compared to the control. Similar to the finding that HE-derived CD45⁺ cells are slower to initiate proliferation (FIG. 3, c), DMK was sufficient for their derivation from HE even in the absence of nucleosides. Thus, a boost in the TCA cycle favors the formation of CD45⁺ mature hematopoietic cells. As observed with similar levels of CD43⁺GPA⁺ as the control in the DMK⁺/nucleosides⁺ condition (FIG. 4, d), this rules out the possibility that CD43⁺CD45⁺ cells take over the cultures to replace other populations. To understand whether DMK induces the formation of definitive hematopoietic cells, day 3 HE cells were co-cultured with OP9-DL1 stroma and induced lymphoid differentiation. While glutamine deprivation or nucleoside supplementation alone during the 3-day subculture prevented HE cells from giving rise to NK cells in co-cultures, DMK or DMK/nucleosides supplementation allowed efficient NK cell differentiation (FIG. 3, e and FIG. 11, k). Thus, glutamine is essential during EHT and differentially regulates the expansion of primitive GPA⁺ and definitive CD45⁺ populations from HE.

Modulation of Pyruvate May Reshape Hematopoietic Output from HE

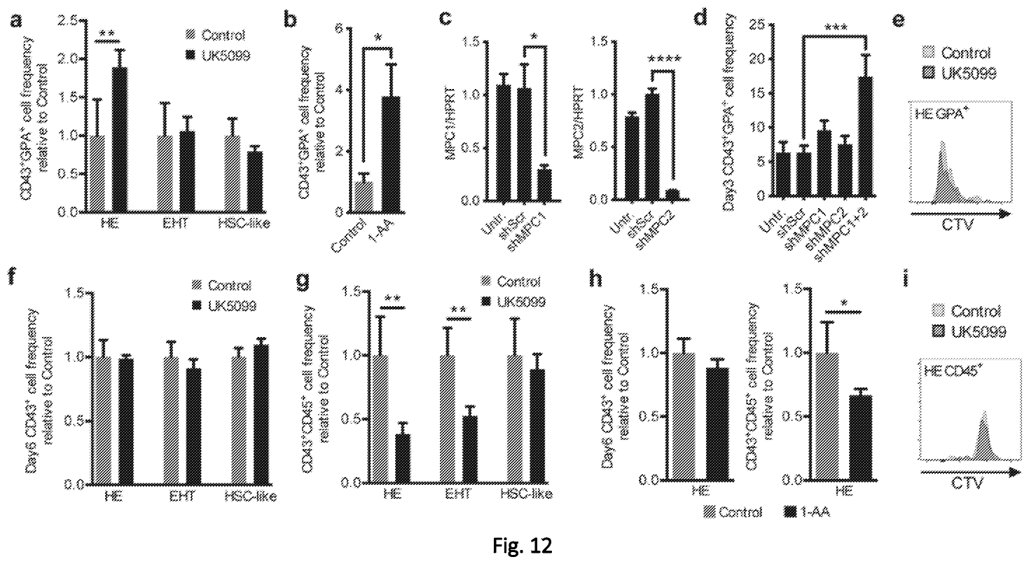

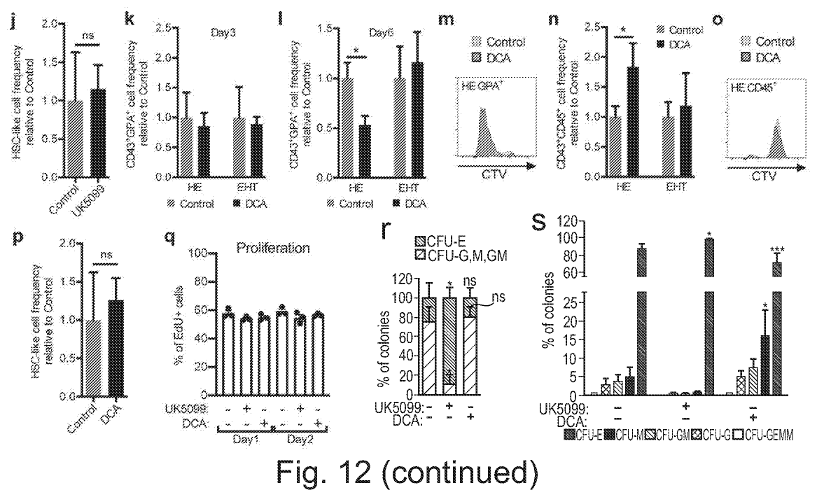

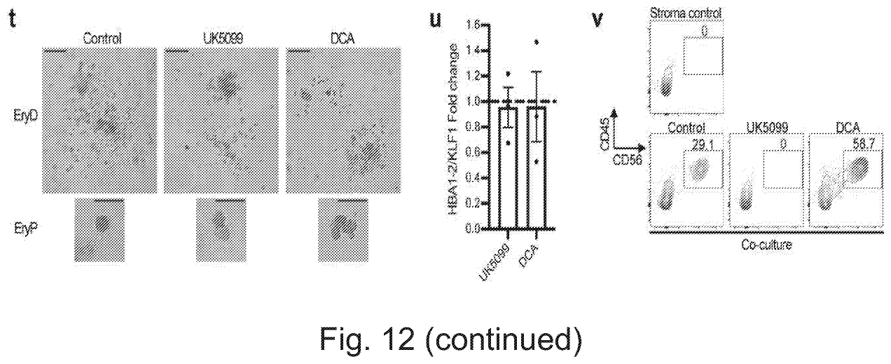

In some examples, HE cells take up glucose at similar levels as EHT cells (FIG. 2f) even though their glycolytic rates are lower, therefore it was investigated whether pyruvate oxidation is important for the hematopoietic commitment of HE cells. Pyruvate is taken up by mitochondria via the mitochondrial pyruvate carrier complex (MPC) and can be converted to acetyl-coA by the PDH enzyme to replenish the TCA cycle (FIG. 4, a). Pyruvate entry into mitochondria was blocked using a specific MPC inhibitor called UK5099 (FIG. 4, a). In HE cells, unlike in EHT or HSC-like cells, MPC inhibition led to a striking increase in CD43⁺GPA⁺ cell output at day 3 of subculture (FIG. 4, b and FIG. 12, a). To confirm this result, HE cells were also treated with 1-aminoethylphosphinic acid (1-AA), a PDH inhibitor (5) (FIG. 4, a) and a significant increase in GPA⁺ cell output was observed compared to the control (FIG. 12, b). Furthermore, in some examples, both MPC subunits, MPC1 and MPC2, were downregulated using shRNAs (FIG. 12, c) and a 2.7-fold increase in CD43⁺GPA⁺ cell output at day 3 of subculture was observed (FIG. 12, d), confirming the results with UK5099. No differences in the proliferation of the GPA⁺ population deriving from HE in the presence of UK5099 as compared to the control was observed (FIG. 12, e). These results demonstrate that the use of glucose for glycolysis may be sufficient to drive erythroid cell formation and that inhibiting pyruvate entry into mitochondria leads to an increased differentiation of HE cells toward the erythroid lineage.

Although the levels of total CD43⁺ cells were unchanged between UK5099-treated and untreated conditions at day 6 (FIG. 12, f), a 2-fold decrease in the CD43⁺CD45⁺ populations deriving from both HE and EHT cells was observed, but not from HSC-like cells (FIG. 4, c, FIG. 12, g). Similarly, 1-AA treatment led to a significant decrease in HE-derived CD45⁺ cell population, even though total CD43⁺ cell levels were unchanged (FIG. 12, h). In some examples, UK5099 did not have an effect on the proliferation of CD45⁺ cells or on the frequency of HSC-like cells, both deriving from HE (FIG. 12, i and j). These results may indicate that blocking pyruvate entry into mitochondria impairs differentiation towards a CD45⁺ hematopoietic fate during EHT.

In certain examples, the opposite effect may be induced by increasing pyruvate flux into mitochondria. Using DCA, PDKs were blocked which repress the PDH complex: this allows pyruvate to be converted to acetyl-coA and potentially fuel the TCA cycle (FIG. 4, a). Although the formation of CD43⁺GPA⁺ cells was not significantly altered by DCA at day 3 of HE subculture (FIG. 12, k), a 50% decrease in this population at day 6 was observed in the treated condition (FIG. 4, d, FIG. 12, l). Thus, as DCA does not directly block glycolysis, it may not affect primitive erythroid differentiation from HE cells. Indeed, the proliferation of the GPA⁺ population deriving from the HE subculture was not affected by DCA at day 3 of subculture (FIG. 12, m). At day 6 of subculture with DCA, an 80% increase in the percentage of CD43⁺CD45⁺ cells deriving from HE was observed, but not EHT cells (FIG. 4, e, FIG. 12, n). The proliferation of CD45⁺ cells or the frequency of HSC-like cells deriving from HE was not affected by DCA (FIG. 12, o and p). To completely rule out any effect of UK5099 and DCA on proliferation, EdU incorporation was performed at early time points of subculture (days 1 and 2) and found no differences in proliferation due to UK5099 or DCA treatments (FIG. 12, q). Taken together, these results show that when pyruvate entry into the TCA cycle is inhibited, HE cells may preferentially give rise to erythroid cells; on the other hand, if pyruvate is pushed towards oxidation in mitochondria, the increased TCA cycle fueling favors definitive CD45⁺ differentiation of HE cells.

In examples, following 3 or 6 days of MPC inhibition with UK5099 in HE cells, erythroid colony (CFU-E) formation was significantly increased compared to the untreated condition, while granulocyte and macrophage colonies were decreased (CFU-G, GM and M) (FIG. 12, r and s). In contrast, while a 3-day PDK inhibition with DCA did not have an effect on CFUs (FIG. 12, r), a 6-day DCA treatment led to a decrease in CFU-Es and a significant increase in CFU-M colonies (FIG. 12, s). The ratio of CFU-E colonies to the sum of CFU-G, CFU-GM and CFU-M colonies was 20-fold higher in UK5099-treated cells and more than 3-fold lower in DCA-treated cells, as compared to the control (FIG. 4, f). In all conditions, both bright red primitive (EryP) and brownish definitive erythroid (EryD) colonies were observed (FIG. 12, t). However, while UK5099 or DCA did not have an effect on HBA1-2 (adult globin) expression (FIG. 12, u), a significant increase in HBE1 (embryonic) and HBG1-2 (fetal) globin transcripts in colonies obtained from UK5099-treated HE cells (FIG. 4, g) was observed, confirming that MPC inhibition increases generation of primitive erythroid cells.

In certain examples, to understand whether DCA induces the formation of definitive hematopoietic cells, lymphoid differentiation was induced in day 3 HE cells in OP9-DL1 stroma co-cultures. While UK5099 treatment impaired NK cell formation, DCA treatment significantly increased NK cell differentiation as compared to untreated HE cells (FIG. 4, h, FIG. 12, v). Altogether, in examples, these results confirm the flow cytometry data and show that while UK5099 may increase primitive erythropoiesis, DCA favors myeloid/lymphoid differentiation from HE at later stages.

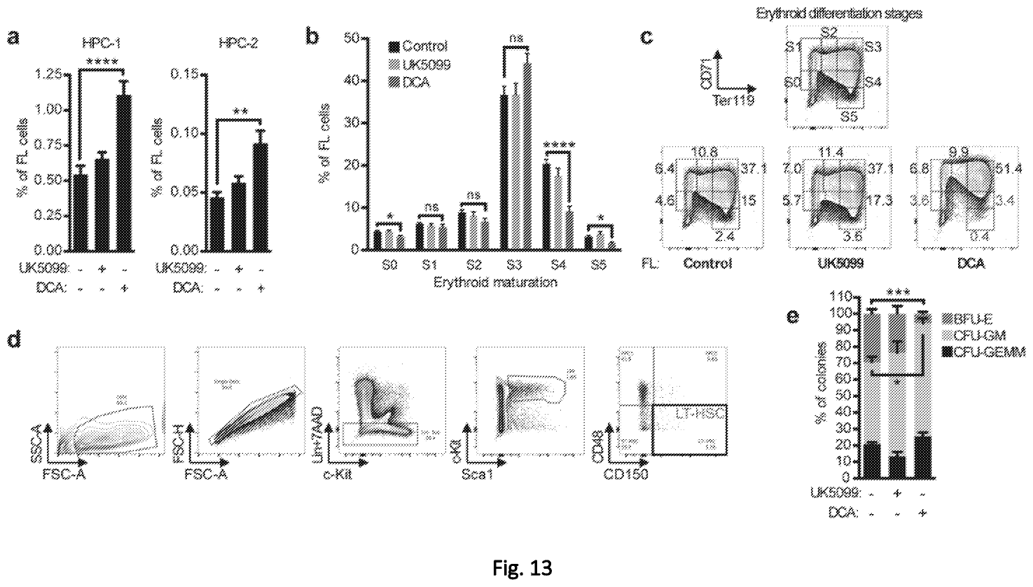

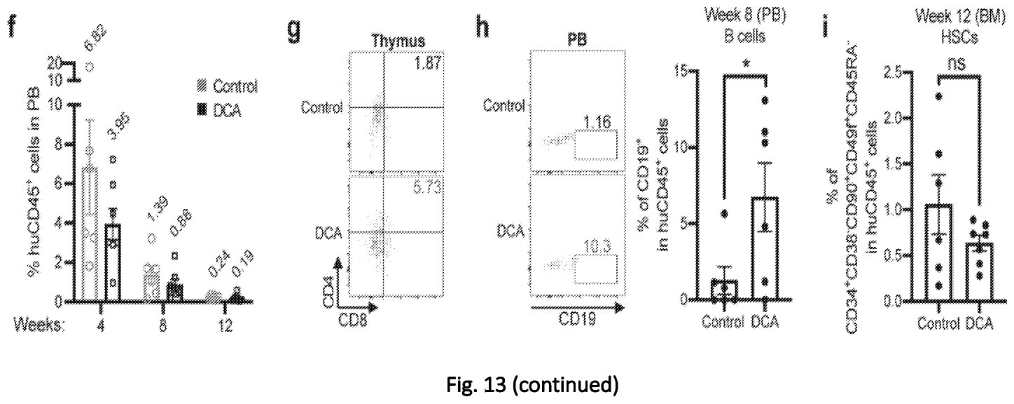

To verify these findings in an in vivo setting, pregnant mice were injected with UK5099 or DCA at embryonic day (E) 9.5, to influence hemogenic endothelium which gives rise to definitive hematopoiesis (both the second and third waves) occurring at E9-9.5 and E10.5, but not primitive hematopoiesis which takes place at E7-7.25 (Palis, J. et al. Development 126, 5073-5084 (1999); and Medvinsky, A. & Dzierzak, E. Cell 86, 897-906 (1996)). In some examples, the blood lineage output in embryos was assessed by characterizing the cellular composition of fetal liver (FL) at E14.5 when the FL is the prime site of hematopoiesis. In some examples, the frequency of phenotypic long-term HSCs (LT-HSCs) were not affected by UK5099 or DCA (FIG. 4, i), confirming the in vitro findings (FIG. 12, j and p). Hematopoietic progenitor cells (HPC)-1, which are restricted progenitors with lymphoid/myeloid potential and HPC-2, which mainly give rise to megakaryocytic progeny were significantly increased in embryos from DCA-injected mice, as compared to the control and UK5099-injected conditions (FIG. 13, a). In line with this, both T and B cell levels were increased in DCA versus control and UK5099-injected embryos (FIG. 4, j), supporting the in vitro results showing an increased CD45⁺ definitive output with DCA. Moreover, in examples, DCA treatment may lead to significant decreases in stage 0, 4 and 5 erythroid populations in the FL, with no significant differences in stages 1, 2 and 3 as compared to the control and UK5099 conditions (FIG. 13, b and c). In some examples, this profile suggests an impairment in definitive erythroid cell production (decrease in S0), while primitive erythrocytes that have formed prior to the injection are in late maturation stages in the FL (S1, 2 and 3) or have exited from the FL into the circulation (decrease in S4 and 5), as described previously (Fraser, S. T. et al. Blood 109, 343-352 (2007); and Isern, J. PNAS 105, 6662-6667 (2008)).

Furthermore, in examples, LT-HSCs from DCA-treated embryos, sorted according to the gating strategy indicated in FIG. 13, d, gave rise to significantly more CFU-GM colonies and less BFU-E colonies (FIG. 13, e), with an 80% decrease in the BFU-E to CFU-GM ratio (FIG. 4, k), as compared to the control and UK5099-treated conditions. No significant effect on in vivo EHT and hematopoiesis by UK5099 (FIG. 4, i-k) was observed, confirming that MPC inhibition preferably affects the primitive hematopoietic wave. Thus, analogous to the results in vitro, PDK inhibition by DCA increases the frequency of lymphoid/myeloid cells at the expense of mature erythroid cells in vivo.

In certain examples in order to assess definitive hematopoietic potential of iPS-derived cells, 3-day DCA-treated HE cells co-cultured with OP9-DL1 stroma were intravenously injected into irradiated NSG mice. Engraftment levels comparable to previous studies were obtained (Rahman, N. et al. Nat Commun 8, 1-12 (2017)), with around 1% human CD45⁺ cells in the peripheral blood (PB) at week 8 (FIG. 13, f). Significantly more human B cells were detected in the PB of NSG mice injected with DCA-treated cells at week 8 (FIG. 13, g), while myeloid cell levels were similar to the untreated condition (FIG. 12, h). At week 12, while similar levels of human HSCs were found (defined as CD34⁺CD38⁻ CD90⁺CD49f⁺CD45RA⁻) in both conditions (FIG. 4, l), a significant increase in the common lymphoid progenitor (CLP) population in the DCA-treated condition (FIG. 4, m) was detected. In line with this result, significantly more human B cells in the BM of NSG mice injected with DCA-treated HE cells (FIG. 4, n) was observed, and no difference in the levels of myeloid cells (FIG. 4, o). Furthermore, significantly more CD4⁺CD8⁺ DP thymocytes in the thymi of NSG mice injected with DCA-treated HE cells (FIG. 4, p, FIG. 13, i) were detected. Taken together, these results may show that increasing pyruvate flux into mitochondria with DCA pushes HE cells towards a definitive hematopoietic and preferentially lymphoid fate in vivo.

### Pyruvate Fate May Dictate Hematopoietic Lineage Commitment of HE Cells at the Single-Cell Level

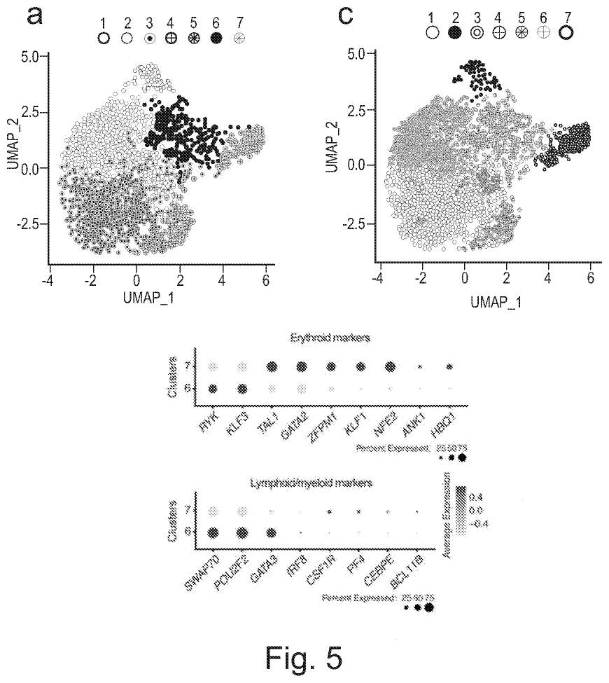

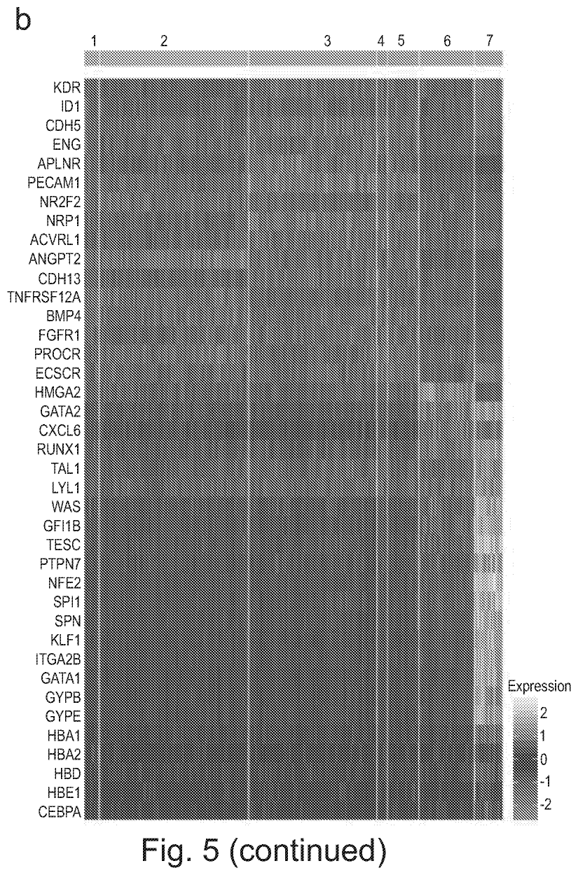

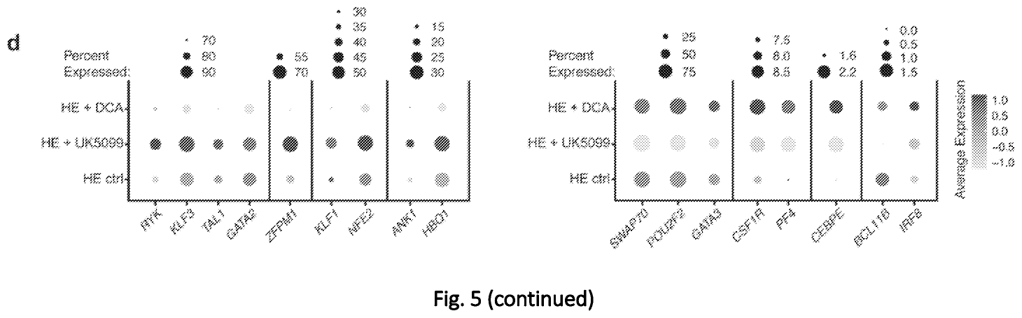

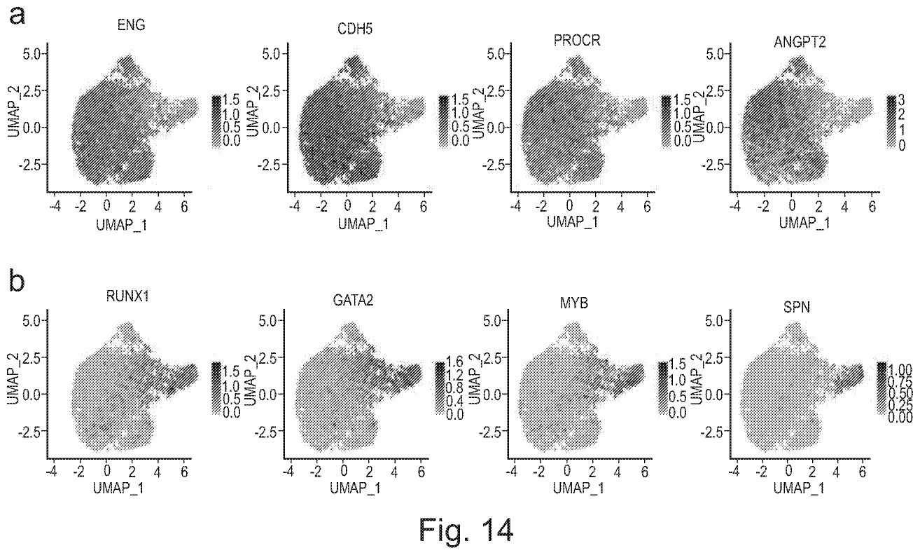

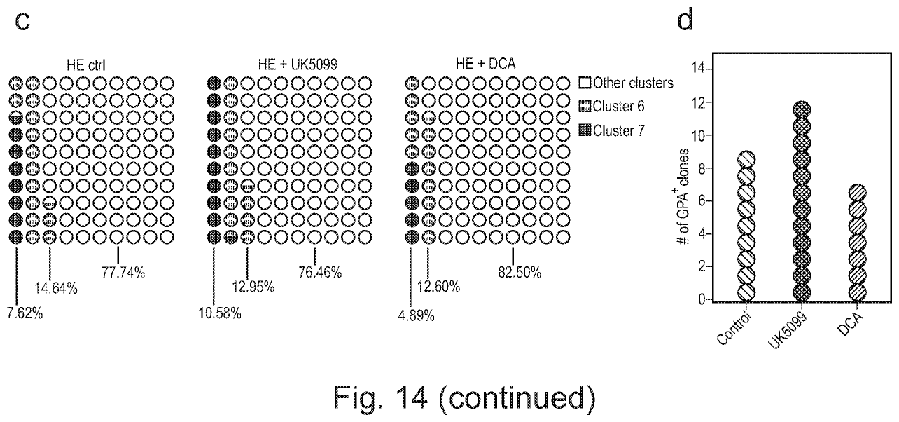

In certain examples, to dissect the molecular effects of pyruvate manipulation, the transcriptmic profiles of HE cells were assessed at an early time point of treatment (day 2), at the single cell level, in control and UK5099- or DCA-treated cells. First, all conditions were grouped together and separated the cells into 7 clusters (FIG. 5, a). The majority of HE cells expressed endothelial markers including ENG, CDH5, PROCR and ANGPT2 (FIG. 14, a) and their expression was mostly confined to clusters 1 through 5 (FIG. 5, b). In contrast, cells in clusters 6 and 7 expressed hematopoietic genes including RUNX1, GATA2, MYB and SPN (FIG. 5, b and FIG. 14, b). Thus, this time point may capture the commitment of HE cells to hematopoietic cells which occurs within clusters 6 and 7.

In examples focusing on isolated clusters 6 and 7 (FIG. 5, c), it was found that an early erythropoiesis regulator, RYK (Tusi, B. K. et al. Nature 555, 54-60 (2018)), and erythroid-specific KLF3, were already expressed in cluster 6 at high levels, while other erythroid markers such as TAL1, GATA2, ZFPM1, KLF1, NFE2, ANK1 and HBQ1 were more highly expressed in cluster 7 (Erythroid markers; FIG. 5, c). Early lymphoid cell fate regulators POU2F2 (B cell) and GATA3 (T cell) as well as myeloid markers SWAP70 and IRF8 were expressed at higher levels in cluster 6, while T lymphoid BCL11B, myelo-monocytic CSF1R, CEBPE, and megakaryocytic PF4 were highest in cluster 7 (Lymphoid/myeloid markers; FIG. 5, c). Thus, while cluster 6 cells expressed early regulators of specific lineages, cluster 7 cells started expressing transcription factors characteristic of more mature hematopoietic cells. Moreover, in cluster 7, the percentage of cells expressing erythroid transcription factors was more than 75%, while cells expressing lymphoid or myeloid markers represented less than 20% of total (FIG. 5, c), in accordance with the early and late emergence of GPA⁺ and CD45⁺ cells, respectively, from HE.

In some examples, while the percentage of cells in cluster 6 was constant between conditions, there were 38% more UK5099-treated HE cells and 35% less DCA-treated HE cells in cluster 7 compared to the control (FIG. 14, c). This result shows that pyruvate modulation may not affect early hematopoietic commitment (cluster 6). However, it seems to have an effect on lineage commitment (cluster 7).

In certain examples, in clusters 6 and 7, the average expression levels of erythroid lineage genes RYK, KLF3, TAL1, GATA2, ZFPM1, KLF1, NFE2, ANK1 and HBQ1 were higher in UK5099-treated HE cells as compared to the untreated HE cells and these factors were nearly absent in DCA-treated HE cells (left-hand dot plot, FIG. 5, d). In contrast, DCA-treated HE cells expressed higher levels of lymphoid/myeloid transcription factors SWAP70, POU2F2, GATA3, CSF1R, PF4, BCL11B, CEBPE and IRF8 compared to control and UK5099-treated HE cells (right-hand dot plot, FIG. 5, d).

In some examples to further assess the effect of pyruvate manipulation on a single cell level, single HE cells were sorted onto OP9-DL1 stroma and GPA⁺ clones were scored at day 14. From a total of 552 single cells per condition, 12 GPA⁺ clones were detected in the UK5099-treated condition and 7 GPA⁺ clones in the DCA-treated condition as compared to 9 GPA⁺ clones in the control (FIG. 14, d). This result may confirm the preferential commitment of HE cells to the erythroid lineage in the presence of UK5099. Taken together, these results may show that at early stages of HE differentiation, modulation of pyruvate use directly impacts the expression of lineage-specific transcription factors and guides the lineage commitment of HE cells.

### Primitive Erythroid Commitment During MPC Inhibition May Rely on LSD1

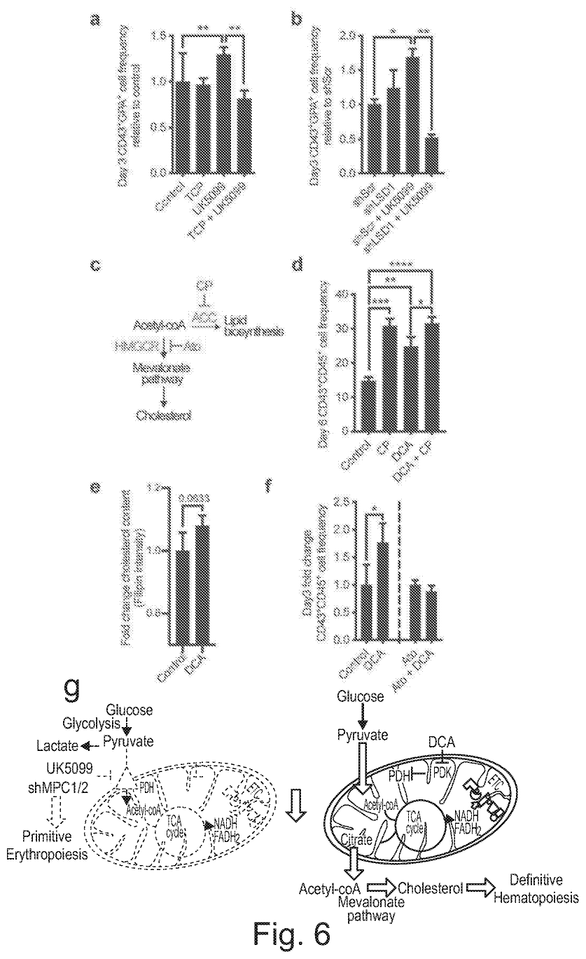

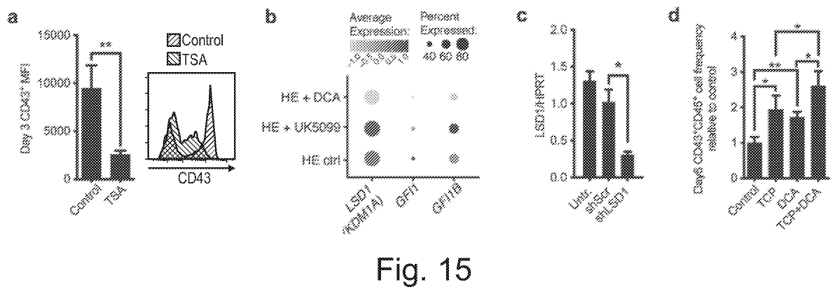

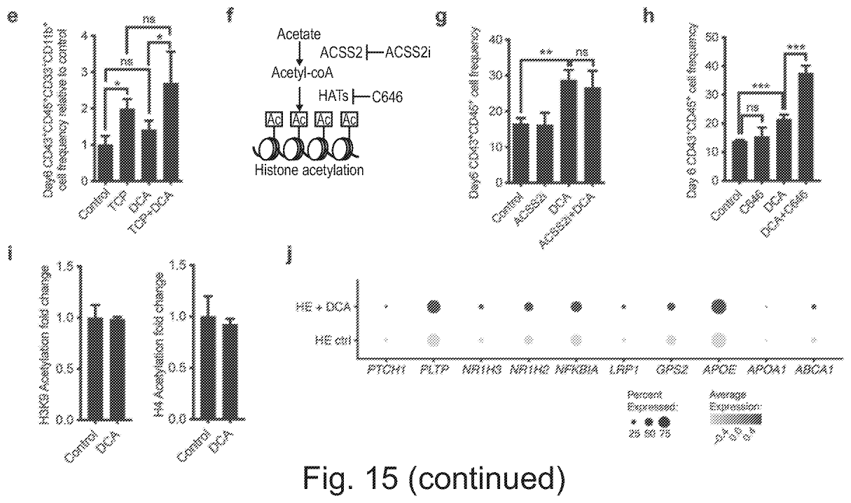

Previous studies have shown that Lysine-Specific Demethylase 1 (LSD1) may be important for EHT and particularly the erythroid lineage (Takeuchi, M. et al. PNAS 112, 13922-13927 (2015); and Thambyrajah, R. et al. Nat Cell Biol 18, 21-32 (2016)). During EHT, LSD1 acts in concert with HDAC1/2 (Thambyrajah, R. et al. Stem Cell Reports 10, 1369-1383 (2018)) and GFI1/GFI1B (Thambyrajah, R. et al. Nat Cell Biol 18, 21-32 (2016)) to induce epigenetic changes. In some examples, it was shown that HDACs may be important for EHT using an HDAC1/2 inhibitor (Trichostatin A, TSA) which impaired the emergence of CD43⁺ hematopoietic cells (FIG. 15, a). Moreover, it was observed that LSD1, GFI1 and GFI1B are expressed at higher levels in UK5099-treated cells as compared to DCA-treated cells (FIG. 15, b), suggesting lineage specification by pyruvate catabolism may be LSD1-dependent. Under conditions where LSD1 was blocked with Tranylcypromine (TCP), or downregulated by shRNAs (FIG. 15, c), no increase in CD43⁺GPA⁺ cell frequency at day 3 following UK5099 treatment of HE cells (FIG. 6, a and b) was detected. On the other hand, TCP-treated HE cells gave rise to more CD43⁺CD45⁺ cells at day 6 similarly to DCA treatment (FIG. 15, d); however, unlike DCA, TCP specifically increased myeloid differentiation (FIG. 15, e), as previously described in the literature (Schenk, T. et al. Nat Med 18, 605-611 (2012)). Thus, mechanistically, the induction of primitive erythropoiesis through MPC inhibition may be dependent on epigenetic regulation by LSD1 in HE cells.

### DCA-Dependent Definitive Hematopoiesis May be Promoted by Cholesterol Metabolism

In some examples, dichloroacetate may be directly used as a precursor of acetylation marks: acetate is converted to acetyl-coA by ACSS2 and transferred onto histones via histone acetyltransferases (HATs) (FIG. 15, f). Inhibiting ACSS2 did not perturb the DCA effect on CD43⁺CD45⁺ cells at day 6 of HE subculture (FIG. 15, g), showing that DCA is not directly converted to acetyl-coA. Moreover, blocking HATs with C646 alone did not have an effect on HE cells; however, C646+DCA treatment boosted the increase in CD43⁺CD45⁺ cells 2-fold compared to DCA alone (FIG. 15, h). As blocking HATs did not inhibit the DCA effect, no changes in global acetylation of H3K9 or H4 with DCA (FIG. 15, i) were found. Thus, inhibiting HATs together with enhancing PDH activity might promote acetyl-coA availability for other metabolic processes, leading to the increase in CD45⁺ cells. Acetyl-coA is a precursor for both lipid biosynthesis (via ACC) and for the mevalonate pathway (via HMGCR) which produces cholesterol (FIG. 6, c). Blocking ACC with CP-640186 (CP) had the same effect as DCA and combined treatment with both CP and DCA further increased the frequency of CD43⁺CD45⁺ cells at day 6 compared to DCA alone (FIG. 6, d). Thus, preventing lipid biosynthesis may increase acetyl-coA availability for cholesterol production. Indeed, in DCA-treated HE cells, an 8% increase in cholesterol content (FIG. 6, e) was detected and higher levels of cholesterol efflux genes at day 2 (FIG. 15, j). Strikingly, treating HE cells with DCA in combination with atorvastatin (Ato) (inhibitor of the mevalonate pathway) abrogated the effect of DCA (FIG. 6, f). Taken together, these results may show that DCA promotes cholesterol biosynthesis which favors definitive hematopoietic commitment of HE cells (FIG. 6, g).

As explained above, during EHT, transitioning cells may go through radical changes in energy use and metabolism, with simultaneous increases in glycolysis and TCA cycle/OXPHOS. The disclosure and results presented herein demonstrate for the first time that glutamine is important for the EHT process, playing distinct roles in the specification of primitive erythroid and definitive hematopoietic cells. On the other hand, in examples, there may be a role for glucose in both glycolysis and the TCA cycle. Blocking its use with 2-DG may impair hematopoietic differentiation of HE cells. In quiescent HSCs, glycolysis was shown to be regulated by hypoxia through the stabilization of hypoxia-inducible factor-1α (HIF-1α) (Takubo, K. et al. Cell Stem Cell 7, 391-402 (2010)). The transition from HE to HSCs was also shown to be regulated by HIF-1α (Harris, J. M. et al. Blood 121, 2483-2493 (2013); and Imanirad, P. et al. Stem Cell Research 12, 24-35 (2014)). Thus, in examples, a HIF-la-dependent induction of glycolysis may be important for EHT.

As shown herein and explained above, glycolysis is sufficient to provide energy for primitive hematopoiesis. Indeed, at early embryonic stages, oxygen is not systemically available and glycolysis is the pathway of choice to produce energy (Gardner, D. K. et al. Semin Reprod Med 18, 205-218 (2000)). In developing embryos, primitive erythroid cells were shown to perform high rates of glycolysis to fuel their rapid proliferation (Baron, M. H. et al. Blood 119, 4828-4837 (2012)). Similarly, in the setting described herein, GPA⁺ cells deriving from HE proliferate faster than CD45⁺ cells and rely on glutamine for providing nucleotides for this process. Likewise, a crucial role for glutamine in supplying nucleotides for erythroid differentiation has been previously described in the context of HSCs obtained from cord blood (Oburoglu, L. et al. Cell Stem Cell 15, 169-184 (2014)). Blocking MPC may redirect HE commitment towards primitive erythropoiesis at a very early stage of EHT, as shown by an increased frequency of committed cells at the single-cell level as well as higher levels of erythroid factors and embryonic/fetal-specific globins in this condition.

In some examples, the results herein and shown above may unravel a role for the TCA cycle and OXPHOS in specifying definitive hematopoietic identity. Fueling the TCA cycle with DMK during glutamine deprivation or DCA treatment may lead to an increased differentiation of HE cells toward a definitive CD45⁺ lineage. While PDK inhibition with DCA does not affect primitive erythroid cell formation, it may induce lymphoid/myeloid-biased definitive hematopoiesis, as shown herein both in vitro and in vivo. DCA-treatment of HE cells may lead to an increased lymphoid reconstitution in NSG mice. The results presented herein are in agreement with previous findings in Pdk2/Pdk4 double knockout mice, which were shown to be anemic but retained normal frequencies of T, B and myeloid populations (Takubo, K. et al. Cell Stem Cell 12, 49-61 (2013)). In examples, the results herein show that DCA may promote CD45⁺ cell formation by fueling cholesterol biosynthesis. This result is corroborated by an elegant study in zebrafish demonstrating that Srebp2-dependent regulation of cholesterol biosynthesis is essential for HSC emergence (Gu, Q. et al. Science 363, 1085-1088 (2019)). As shown here, a direct metabolic change in HE cells, namely increased acetyl-coA content, can promote cholesterol metabolism and control definitive hematopoietic output.

Others have reported previously that distinct EHT cell subsets or pre-HSCs present different lineage propensities (Zhou, F. et al. Nature 533, 487-492 (2016); and Guibentif, C. et al. Cell Reports 19, 10-19 (2017)). In certain examples such as shown herein, metabolism can rewire the fate of HE cells, suggesting that lineage propensities may be decided at the HE level. In line with the results herein, a recent study combining scRNAseq with lentiviral lineage tracing revealed that cell fate biases appear at a much earlier stage during hematopoietic development than previously described with conventional methods (Weinreb, C. et al. Science. 2020 Feb. 14; 367(6479)). Furthermore, murine HSCs were shown to present lymphoid or myeloid hematopoietic lineage biases due to epigenetic priming which is established prior to their formation (Yu, V. W. C. et al. Cell 167, 1310-1322.e17 (2016)). In effect, linking epigenetic changes to metabolism is a newly emerging field which reconciliates metabolic alterations with transcriptional regulation of cellular processes. In accordance, as shown in examples herein, erythroid fate induction by MPC inhibition may be dependent on an epigenetic factor, LSD1.

The examples and results herein may indicate that the lineage propensities of primitive and definitive hematopoietic waves are shaped by nutrient availability in the YS and AGM niches. Due to scarcity of oxygen in early embryonic stages, the primitive hematopoietic wave may depend on glycolysis to form erythroid cells expressing embryonic globins with high affinity for oxygen (FIG. 6, g). This may allow for an efficient distribution of oxygen to newly forming tissues and promote the use of OXPHOS, which may initiate the emergence of the definitive hematopoietic waves (FIG. 6, g).

As explained herein, in examples, using metabolic determinants to direct definitive HSC development in vitro from PSCs may provide a way to produce transplantable cells, able to reconstitute the hematopoietic system of patients with hematological malignancies and disorders.

hiPSC Culture, Hematopoietic Differentiation and Cell Isolation Methods

One of skill in the art will understand that the methods and materials described below and elsewhere in the specification are merely examples, and such examples may be performed using different combinations of methods and materials. Further, elements of the methods and materials described herein may be optional. The RB9-CB1 human iPSC line was co-cultured with mouse embryonic fibroblasts (MEFs, Millipore), passaged every six days and processed to form embryoid bodies (EBs) as described previously (Guibentif, C. et al. Cell Reports 19, 10-19 (2017)). The differentiation protocol used in this study was previously described (Ditadi, A. & Sturgeon, C. M. Methods 101, 65-72 (2016)), however, small modifications were made to induce both primitive and definitive hematopoiesis, as indicated below and in FIG. 7, a. Newly-formed EBs were first kept in SFD medium supplemented with 1 ng/ml Activin A (on days 0-2) and 3 μM CHIR99021 (on day 2 only). At day 3, media was switched to “Day 3-SP34” medium supplemented with 1 ng/ml Activin A (on day 3 only) and 3 μM CHIR99021 (on day 3 only) until day 6. On day 6, media was replaced by “Day 6-SP34” medium, until day 8. In some experiments where indicated, to obtain a higher yield of HSC-like cells, EBs were kept until day 10: in this case, EBs were plated onto Matrigel (8 μg/cm², Corning)-coated dishes on day 8 and kept until day 10. Media was changed every day, except on days 5 and 7. On day 8 or 10 (as indicated), EBs were singularized with 5-6 rounds of 5-minute incubations with TryPLE Express (Thermo Fisher Scientific). CD34⁺ cells were selected using the human CD34 MicroBead kit (Miltenyi Biotec) and stained with CD34-FITC, CD73-PE, VECad-PerCPCy5.5, CD38-PC7, CD184-APC, CD45-AF700, CD43-APCH7, GPA-eF450, CD90-BV605 and the viability marker 7AAD in order to sort HE (CD34⁺CD43⁻CXCR4⁻CD73⁻CD90⁺VECad⁺), EHT (CD34⁺CD43^{int}CXCR4⁻CD73⁻CD90⁺VECad⁺) and HSC-like (CD34⁺CD43⁺CD90⁺CD38⁻) cells, according to previously described markers (Guibentif, C. et al. Cell Reports 19, 10-19 (2017); Harris, J. M. et al. Blood 121, 2483-2493 (2013) and Schenk, T. et al. Nat Med 18, 605-611 (2012)).

### HE, EHT and HSC-Like Subculture

Sorted HE (40,000), EHT (30,000) and HSC-like (5-20,000) cells were plated onto Matrigel (16 μg/cm², Corning)-coated 96-well flat bottom plates in HE medium (30) with 1% penicillin-streptomycin and kept in a humidified incubator at 37° C., 5% CO2, 4% O₂ overnight. The following day (day 0), wells were washed twice with PBS and fresh HE medium was added, together with 2-DG (1 mM), UK5099 (10 μM), DCA (3 mM), BPTES (25 μM), TSA (60 nM), TCP (300 nM), ACSS2i (5 μM), C646 (10 μM), CP-640186 (5 μM), Atorvastatin (0.5 μM), or in glutamine-free medium with DMK (1.75 mM), nucleosides (1×) or NEAAs (1×), where indicated. Media was changed and drugs were added every 2 days and cells were kept in a humidified incubator at 37° C., 5% CO2, 20% O₂ for 6-7 days. Pictures were taken using an Olympus IX70 microscope equipped with a CellSens DP72 camera and CellSens Standard 1.6 software (Olympus).

### Extracellular Flux Analyses

For comparisons between HE, EHT and HSC-like cells, day 10 FACS-sorted cells (≥40,000) were directly plated onto Seahorse XF96 Cell Culture Microplate wells coated with CellTak (0.56 μg/well) in 2-4 replicates and extracellular flux was assessed immediately on a Seahorse XF96 analyzer. For comparisons between HE and EHT cells, day 8 FACS-sorted cells (≥40,000) were plated onto Matrigel (16 μg/cm², Corning)-coated Seahorse XF96 Cell Culture Microplate wells in 3-4 replicates and extracellular flux was assessed 2 days after plating, on a Seahorse XF96 analyzer. To assess glycolytic flux, ECAR was measured in XF medium with 2 mM glutamine under basal conditions (after 1-hour glucose starvation as per manufacturer's instructions) as well as after the addition of 25 mM glucose, 4 μM oligomycin and 50 mM 2-DG and data was normalized to cell number. The levels of glycolytic capacity (ECAR_{oligomycin}-ECAR_{2-DG}) and glycolysis (ECAR_{glucose}-ECAR_{2-DG}) were calculated. To assess oxidative phosphorylation, OCR was measured in XF medium with 10 mM glucose, 2 mM glutamine and 1 mM sodium pyruvate under basal conditions as well as after the addition of 4 μM oligomycin, 2 μM Carbonyl cyanide 4-(trifluoromethoxy)phenylhydrazone (FCCP) and 1 μM rotenone/40 μM Antimycin A and data was normalized to cell number. The levels of basal respiration (OCR_{basal}-OCR_{Rotenone/AntimycinA}), ATP production (OCR_{basal}-OCR_{Oligomycin}) and maximal respiration (OCR_{FCCP}-OCR_{Rotenone/AntimycinA}) were calculated.

### Flow Cytometry Analyses

On days 3 and 6 of subculture, cells were collected after a 2-minute incubation at 37° C. with StemPro Accutase Cell Dissociation Reagent and stained with CD34-FITC, CD14-PE, CD33-PC7, CD11b-APC, CD45-AF700, CD43-APCH7, GPA-eF450, CD90-BV605 and the viability marker 7AAD and fluorescence was measured on a BD LSRII. To measure mitochondrial activity, cells were incubated with Tetramethylrhodamine ethyl ester (TMRE, 20 nM) for 30 minutes at 37° C. Negative controls were incubated with 100 μM FCCP for 30 minutes at 37° C., prior to TMRE staining. Fluorescence was measured on a BD FACSARIA III and MFI levels—MFI FMO were calculated. To measure glucose uptake, cells were incubated with 2-(N-(7-Nitrobenz-2-oxa-1,3-diazol-4-yl)Amino)-2-Deoxyglucose (2-NBDG) for 30 minutes at 37° C. and fluorescence was measured on a BD FACSARIA III. To measure proliferation, cells were processed with the CellTrace Violet (CTV) kit according to manufacturer's instructions (10-minute incubation) and fluorescence was measured on a BD LSRFortessa. To measure EdU incorporation, HE cells were assessed on day 1 or 2 of subculture after 24 h EdU pulses, using Click-iT EdU Flow Cytometry Cell Proliferation Assay (Thermo Fisher Scientific, C10424), according to manufacturer's instructions. Flow cytometry outputs were analyzed on FlowJo Software, with initial gatings on SSC-A/FSC-A, FSC-H/FSC-A, SSC-H/SSC-A and 7-AAD to exclude doublets and dead cells in all experiments.

### Colony Forming Unit Assay

Subcultured HE cells were treated with StemPro Accutase Cell Dissociation Reagent for 2 minutes at 37° C. and dissociated cells were resuspended in 3 ml Methocult H4230 (STEMCELL Technologies, France) (prepared according to manufacturer's instructions, with 20 mL Iscove's Modified Dulbecco's Medium containing 2.5 μg hSCF, 5 μg GM-CSF, 2.5 μg IL-3 and 500 U EPO). Each mixture was divided onto 2 wells of a non-tissue culture treated 6-well plate. Following a 12-day incubation in a humidified incubator at 37° C., 5% CO2, 20% O₂, colonies were morphologically distinguished and scored. For globin analysis, colonies in Methocult wells were harvested with PBS, washed thoroughly and frozen in RLT buffer with β-mercaptoethanol. Following RNA extraction and RT (Qiagen), gene expression was assessed with taqman probes by q-PCR. The taqman probes used in this study are HBA1/2 (Hs00361191_g1), HBE1 (Hs00362216_m1), HBG2/1 (Hs00361131_g1) and KLF1 (Hs00610592_m1).

### Lymphoid Differentiation Assay on OP9-DL1 Stroma

Subculture day 3 HE cells cultured in the presence of UK5099 (10 μM), DCA (3 mM), or in glutamine-free medium with DMK (1.75 mM), nucleosides (1×) or NEAAs (1×), as indicated, were collected after a 2-minute incubation at 37° C. with StemPro Accutase Cell Dissociation Reagent and seeded onto 80% confluent OP9-DL1 stroma. Cells were cultured in OP9 medium with SCF (10 ng/ml), FLT3-L (10 ng/ml), IL-2 (5 ng/ml), IL-7 (5 ng/ml, first 15 days only) and IL-15 (10 ng/ml) with passaging onto new OP9-DL1 stroma every week, as described previously (Renoux, V. M. et al. Immunity 43, 394-407 (2015)). At day 35 of co-culture, cells were analyzed on a BD LSRFortessa.

### Single-Cell RNAseq Library Preparation and Sequencing

Sorted HE, EHT and HSC-like cells as well magnetically selected (Miltenyi Biotec) cord blood CD34⁺ cells were plated onto Matrigel (16 μg/cm², Corning)-coated 96-well flat bottom plates in HE medium (Ditadi, A. & Sturgeon, C. M. Methods 101, 65-72 (2016)) with 1% penicillin-streptomycin and kept in a humidified incubator at 37° C., 5% CO2, 4% O₂ overnight. The following day (day 0), wells were washed twice with PBS and fresh HE medium was added, together with UK5099 (10 μM) or DCA (3 mM), where indicated. On day 1 and 2 (as indicated), cells were washed twice with PBS 0.04% UltraPure BSA and collected after a 2-minute incubation at 37° C. with StemPro Accutase Cell Dissociation Reagent. Cells were spun down, resuspended in PBS 0.04% UltraPure BSA, counted (yield between 8,000-18,000 cells) and library preparation was conducted according to the Chromium Single Cell 3′ Reagent kit v3 instructions (10× Genomics). Sequencing was performed on a NOVASeq 6000 from Illumina with the run parameters (28-8-0-91) recommended by 10× Genomics with a final loading concentration of 300 pM of the pooled libraries. Human Umbilical Cord Blood samples were collected from Skane University Hospital (Lund and Malmo) and Helsingborg Hospital with informed consents according to guidelines approved by the regional ethical committee.

### Single-Cell RNAseq Analysis

The data was processed and analyzed using Seurat v3.1.0, where cells were allowed to have up to 20% mitochondrial reads prior to log-normalization and finding the top 500 variable genes using the “vst” method. Cell cycle scores were calculated and the data was scaled regressing on mitochondrial content and the difference of the S and G2M score. Principal components were calculated prior to calculating a UMAP. Pseudotime trajectories describing two developmental routes were identified in our EHT dataset using Slingshot (Street, K. et al. BMC Genomics 19, 477 (2018)) along which the cells were ordered. The cells were then binned along each trajectory where the cell-type composition of each bin was calculated as percentages. Cord blood CD34⁺ cells were mapped to our data and labeled using scCoGAPS (Stein-O'Brien, G. L. et al. Cell Syst 8, 395-411.e8 (2019)). CS13 data from Zeng et al. (Zeng, Y. et al. Cell Res 1-14 (2019)) was read and processed to make a UMAP from which the cells they name as “AEC” and “Hem” were identified. These 99 cells were mapped to our data and labeled using SCMAP (Kiselev, V. Y. et al. Nat Methods 15, 359-362 (2018)). Our EHT data was mapped to data from Zeng et al. (Zeng, Y. et al. Cell Res 1-14 (2019)) and vice versa using scCoGAPS where 10 patterns were identified in each data set and then projected to each other using projectR. Each cell was assigned to the group that achieved the highest weight. An overview showing the relationship between cell-types and patterns was done by forming a contingency table on which correspondence analysis was performed using the ca package for R. Differentially expressed genes were found using the FindAllMarkers function. Cell numbers for day 1 samples are as follows: HE=1451, EHT=1523, HSC-like=732. Cell numbers for day 2 samples are as follows: HE ctrl=1195, HE+UK5099=718, HE+DCA=2309. All assessed endothelial and hematopoietic genes were previously used in several publications to validate the EHT process (Zhou, F. et al. Nature 533, 487-492 (2016); Swiers, G. et al. Nat Commun 4, 2924 (2013); Ng, E. S. et al. Nature Biotechnology 34, 1168-1179 (2016) and Guibentif, C. et al. Cell Reports 19, 10-19 (2017)). For gene expression analyses, gene sets for glycolysis, oxidative phosphorylation, glutamine transport and cholesterol efflux were downloaded from The Molecular Signatures Database (MSigDB).

Downregulation Via shRNAs

Short-hairpin sequences recognizing the genes of interest were cloned into GFP-expressing pRRL-SFFV vectors, embedded in a microRNA context for minimal toxicity, as described previously (Fellmann, C. et al. Cell Reports 5, 1704-1713 (2013)). Each lentivirus batch was produced in two T175 flasks of HEK 293T cells by co-transfecting 22 μg of pMD2.G, 15 μg of pRSV-Rev, 30 μg of pMDLg/pRRE and 75 μg of the shRNA vector using 2.5 M CaCl₂. Medium was changed 16 hours after transfection and viruses were harvested 48 hours after transfection, pelleted at 20,000×g for 2 hours at 4° C., resuspended in 100 μl DMEM, aliquoted and kept at −80° C. The downregulation efficiency of each shRNA was measured by assessing the corresponding gene expression by qPCR in sorted GFP⁺ cells, 3 days after lentiviral transduction of cord blood CD34⁺ HSPCs. HE cells were transduced by direct addition of lentivirus particles into the culture medium on the day after the sort.

### In Vivo Compound Injections and Murine Hematopoiesis Assessment

Pregnant female C57Bl/6xB6.SJL mice were injected intraperitoneally at E9.5 with UK5099 (4 mg/kg) or DCA (200 mg/kg) or PBS (control). Embryos were harvested at E14.5 and individually weighed and processed. Fetal livers were dissected and homogenized in 800 μL ice cold PBS supplemented with 2% fetal bovine serum (FBS) and FL cells were washed in PBS with 2% FBS. For the differentiated lineage panel, cells were stained with B220 and CD19 (B-cell markers)-PE, CD3e-APC, Ter119-PeCy7 and CD71-FITC and analyzed on a BD FACSARIA III. For the HSC panel, samples were first treated with ammonium chloride solution (STEMCELL Technologies, France) to lyse red blood cells, washed twice in ice cold PBS with 2% FBS, stained with CD3e, B220, Ter119, Gr1 (Lineage)-PeCy5, c-Kit-Efluor780, Sca1-BV421, CD48-FITC, CD150-BV605 and 7-AAD (for dead cell exclusion) and analyzed on a BD FACSARIA III. Flow cytometry outputs were analyzed on FlowJo Software, with initial gatings on SSC-A/FSC-A and FSC-H/FSC-A to exclude doublets. For plating of CFU assays, 100 LT-HSCs were sorted (gating strategy shown in FIG. 13, d) and resuspended in 3.0 mL Methocult M3434 (STEMCELL Technologies, France). Each mixture was divided onto 2 wells of a non-tissue culture treated 6-well plate. Following a 14-day incubation in a humidified incubator at 37° C., 5% CO₂, 20% O₂, colonies were morphologically distinguished and scored.

### NSG Mice Transplantations

Sorted human HE cells (350,000) were mixed with OP9-DL1 stroma (60,000) and subcultured for 3 days with or without DCA (3 mM) on Matrigel (16 μg/cm², Corning)-coated 12-well plates in HE medium³⁰. Between 100,000-150,000 cells from control or DCA samples were transplanted into sub-lethally irradiated (300 cGy) 8-week-old female NOD/Cg-Prkdc^{scid} Il2rg^{tm1Wjl}/SzJ mice (NSG, The Jackson Laboratory) together with 20,000 whole bone marrow support cells from C57Bl/6.SJL mice (CD45.1+/CD45.2+, in house breeding). Cells were transplanted in single cell solution in 250 μL PBS with 2% FBS through intravenous tail vein injection. Drinking water of transplanted NSG mice was supplemented with ciprofloxacin (125 mg/L, HEXAL) for 3 weeks after transplantation to prevent infection. Mice were housed in a controlled environment with 12-hour light-dark cycles with chow and water provided ad libitum. Experiments and animal care were performed in accordance with the Lund University Animal Ethical Committee.

### Peripheral Blood Analysis After NSG Mice Transplantations

Peripheral blood (PB) was collected from the tail vein into EDTA-coated microvette tubes (Sarstedt, Cat #20.1341.100). Peripheral blood was lysed for mature erythrocytes in ammonium chloride solution (STEMCELL technologies) for 10 minutes at room temperature, washed and stained for cell surface antibodies for 45 minutes at 4° C., washed and filtered prior to flow cytometry analysis on the FACS AriaIII (BD). Flow cytometry outputs were analyzed on FlowJo Software, with initial gatings on SSC-A/FSC-A and FSC-H/FSC-A for doublet exclusion, on DAPI for dead cell exclusion and on huCD45/muCD45.1 for murine cell exclusion.

### Bone Marrow Analysis After NSG Mice Transplantations

Bone marrow was analyzed at the 12-week transplantation endpoint. Mice were euthanized by spinal dislocation followed by the dissection of both right and left femurs, tibias and iliac bones. Bone marrow was harvested through crushing with a pestle and mortar and cells were collected in 20 mL ice-cold PBS with 2% FBS, filtered and washed (350×g, 5 min). Bone marrow cells were lysed for red blood cells (ammonium chloride solution, STEMCELL technologies) for 10 minutes at room temperature, washed and stained for cell surface antibodies for 45 minutes at 4° C., washed and filtered prior to FACS analysis on the FACS AriaIII (BD). Flow cytometry outputs were analyzed on FlowJo Software, with initial gatings on SSC-A/FSC-A and FSC-H/FSC-A for doublet exclusion, on DAPI or 7AAD for dead cell exclusion and on huCD45/muCD45.1 for murine cell exclusion.

### Thymus Analysis After NSG Mice Transplantations

Whole thymus was harvested at the 12-week transplantation endpoint. The thymocytes were mechanically dissociated from connective tissue in the thymus by pipetting up and down in PBS with 2% FBS, followed by filtration through a 50 μm sterile filter. Erythrocyte contamination was removed by lysing the sample in ammonium chloride solution (STEMCELL technologies) for 10 minutes at room temperature. Samples were washed and spun down after and the pellet of thymocytes was resuspended in FACS buffer and stained for cell surface antibodies for 45 minutes at 4° C., washed and filtered prior to FACS analysis on the FACS AriaIII (BD). Flow cytometry outputs were analyzed on FlowJo Software, with initial gatings on SSC-A/FSC-A and FSC-H/FSC-A for doublet exclusion, on DAPI for dead cell exclusion and on huCD45/muCD45.1 for murine cell exclusion.

### Confocal Microscopy Imaging and Quantification

For TMRE staining, on day 3 of subculture, half of the culture medium was removed and cells were stained with 20 nM TMRE (Thermo Fisher Scientific, T669) by direct addition into the culture medium of a 2× concentrated solution. After a 20-minute incubation at 37° C., wells were carefully washed with PBS and fresh HE medium was added. During acquisition, cells were kept in a humidified incubator at 37° C., 5% CO₂, 20% O₂. For immunocytochemistry, subculture day 2 HE cells (plated on coverslips) were washed twice in PBS, fixed with 4% PFA for 15 minutes at RT and washed three times with PBS. For filipin staining, fixed cells were incubated with 100 μg/ml filipin III (Sigma-Aldrich, F4767) for 1 hour, washed three times with PBS and rinsed with distilled water before mounting with PVA/DABCO. For H3K9 and H4 acetylation staining, fixed cells were permeabilized and blocked 1 hour at RT with PBS+0.25% Triton X-100+5% normal donkey serum (blocking solution) followed by incubation overnight at 4° C. with primary antibodies diluted in blocking solution. Cells were then washed 2×5 min with PBS+0.25% Triton X-100 (TPBS) and 5 min with blocking solution before incubation with secondary antibodies 2 hours at RT diluted in blocking solution. Cells were later washed 5 min with TPBS containing 1 μg/ml Hoechst and twice with PBS before being rinsed with distilled water and mounted with PVA:DABCO. Images were obtained with the 10× (TMRE) or 20× (Filipin and acetylation) objective of a Zeiss LSM 780 confocal microscope using the Zen software and a 1.5× zoom (TMRE) or 0.6× zoom (Filipin and acetylation). Acquisition settings were the same for all images of each experiment, taking the same number of stacks. Intensity quantification was performed using the Fiji software as follows. For TMRE, using the brightfield channel, ROIs were selected for 5 spindle-shaped and 5 round cells (randomly chosen) and average intensity for each ROI was calculated in a summatory Z-stack of the TMRE channel. For filipin and acetylation, the summatory Z-stack for the filipin channel was obtained and average intensity calculated. A total of 2-3 independent experiments with 2-3 replicate wells were quantified. For each replicate well, 4-6 images were acquired.

### Statistical Analyses

Significance of differences between conditions were calculated using paired/unpaired t-tests, 1/2-way analysis of variance (ANOVA) tests or Kruskal-Wallis tests with multiple comparisons in GraphPad Prism 6 software, as indicated. p values are indicated in figures with the following abbreviations: ns, not significant, *p<0.05, **p<0.01, ***p<0.001, ****p<0.0001.

While the present description sets forth specific details of various embodiments, it will be appreciated that the description is illustrative only and should not be construed in any way as limiting. Furthermore, various applications of such embodiments and modifications thereto, which may occur to those who are skilled in the art, are also encompassed by the general concepts described herein. Each and every feature described herein, and each and every combination of two or more of such features, is included within the scope of the present invention provided that the features included in such a combination are not mutually inconsistent. All figures, tables, and appendices, as well as patents, applications, and publications, referred to above, are hereby incorporated by reference.

Some embodiments have been described in connection with the accompanying drawing. However, it should be understood that the figures are not drawn to scale. Distances, angles, etc. are merely illustrative and do not necessarily bear an exact relationship to actual dimensions and layout of the devices illustrated. Components can be added, removed, and/or rearranged. Further, the disclosure herein of any particular feature, aspect, method, property, characteristic, quality, attribute, element, or the like in connection with various embodiments can be used in all other embodiments set forth herein. Additionally, it will be recognized that any methods described herein may be practiced using any device suitable for performing the recited steps.

For purposes of this disclosure, certain aspects, advantages, and novel features are described herein. It is to be understood that not necessarily all such advantages may be achieved in accordance with any particular embodiment. Thus, for example, those skilled in the art will recognize that the disclosure may be embodied or carried out in a manner that achieves one advantage or a group of advantages as taught herein without necessarily achieving other advantages as may be taught or suggested herein.

Although these inventions have been disclosed in the context of certain preferred embodiments and examples, it will be understood by those skilled in the art that the present inventions extend beyond the specifically disclosed embodiments to other alternative embodiments and/or uses of the inventions and obvious modifications and equivalents thereof. In addition, while several variations of the inventions have been shown and described in detail, other modifications, which are within the scope of these inventions, will be readily apparent to those of skill in the art based upon this disclosure. It is also contemplated that various combination or sub-combinations of the specific features and aspects of the embodiments may be made and still fall within the scope of the inventions. It should be understood that various features and aspects of the disclosed embodiments can be combined with or substituted for one another in order to form varying modes of the disclosed inventions. Further, the actions of the disclosed processes and methods may be modified in any manner, including by reordering actions and/or inserting additional actions and/or deleting actions. Thus, it is intended that the scope of at least some of the present inventions herein disclosed should not be limited by the particular disclosed embodiments described above. The limitations in the claims are to be interpreted broadly based on the language employed in the claims and not limited to the examples described in the present specification or during the prosecution of the application, which examples are to be construed as non-exclusive.

1. A method of generating definitive hematopoietic cells, comprising;
providing a plurality of source cells selected from the group consisting of differentiating iPS cells, cells directly reprogrammed to pre-cursors of hematopoietic cells, cells directly reprogrammed to definitive hematopoietic cells, and adult or neonatal hematopoietic cells derived from bone marrow, cord blood, placenta, or mobilized peripheral blood; and
treating the source cells with a metabolic regulator, the metabolic regulator configured to activate a tricarboxylic acid cycle of the source cells.

2. The method according to claim 1, wherein the metabolic regulator is configured to inhibit pyruvate dehydrogenase kinases (PDK).

3. The method according to claim 1, wherein the metabolic regulator is configured to activate pyruvate dehydrogenase complexes (PDH).

4. The method according to claim 1, wherein the metabolic regulator is configured to increase uptake of pyruvate into mitochondria.

5. The method according to claim 1, wherein the metabolic regulator is configured to accelerate conversion of pyruvate to acetyl coenzyme A (Ac-CoA).

6. The method according to claim 1, wherein the metabolic regulator is dichloroacetate (DCA).

7. The method according to claim 6 wherein the concentration of the dichloroacetate is at least about 30 μM.

8. The method according to claim 7, wherein the DCA is configured to induce lymphoid/myeloid-biased definitive hematopoiesis.

9. The method according to claim 1, wherein the metabolic regulator is an LSD1 inhibitor.

10. The method according to claim 9 wherein the LSD1 inhibitor comprises at least one of GSK2879552 or RO7051790.

11. The method according to claim 9, wherein the LSD1 inhibitor is configured to generate definitive hematopoietic cells of the erythroid lineage.

12. The method according to claim 1 wherein the metabolic regulator is configured to increase production of α-ketoglutarate.

13. The method according to claim 12 wherein the metabolic regulator is glutamine.

14. The method according to claim 12, further comprising generating CD43⁺ cells from a hemogenic endothelial (HE) source cell in response to treatment with the metabolic regulator.

15. The method according to claim 12, further comprising treating the source cells with nucleoside triphosphates.

16. The method according to claim 1, wherein the metabolic regulator is a more potent or more stable equivalent of α-ketoglutarate.

17. The method according to claim 16, wherein the metabolic regulator is dimethyl α-ketoglutarate (DMK).

18. The method according to claim 17, wherein the concentration of Dimethyl α-ketoglutarate is at least about 17.5 μM.

19. The method of claim 16, wherein the metabolic regulator is used in combination with a nucleoside.

20. The method of claim 19, wherein the concentration of nucleoside is at least about 0.7 mg/L.

21. The method of claim 19, wherein the nucleoside comprises a nucleoside selected from the group consisting of cytidine, guanosine, uridine, adenosine, and thymidine.

22. The method according to claim 1, wherein the definitive hematopoietic cells comprise definitive hematopoietic stem cells.

23. The method according to claim 22 wherein the definitive hematopoietic stem cells have lymphoid and/or myeloid repopulating potential.

24. The method according to claim 1, wherein the definitive hematopoietic cells comprise definitive lymphoid and/or myeloid cells.

25. The method of claim 24, wherein where the definitive lymphoid cells comprise cells selected from the group consisting of T-cells, modified T-cells targeting tumor cells, B-cells, NK cells and NKT cells.

26. The method according to claim 1, wherein the definitive hematopoietic cells comprise mast cells.

27. The method according to claim 1, wherein the definitive hematopoietic cells comprise erythroid cells suitable for the production of adult hemoglobin.

28. The method according to claim 1, wherein cells directly reprogrammed to pre-cursors of hematopoietic cells comprise cells selected from the group consisting of mesodermal precursor cells, hemogenic endothelium cells, and cells undergoing endothelial to hematopoietic transition.

29. The method according to claim 1, wherein adult or neonatal hematopoietic cells comprise hematopoietic stem cells or hematopoietic progenitor cells.

30. A method of generating definitive hematopoietic cells, comprising:
providing a plurality of source cells selected from the group consisting of differentiating iPS cells, cells directly reprogrammed to pre-cursors of hematopoietic cells, cells directly reprogrammed to definitive hematopoietic cells, and adult or neonatal hematopoietic cells derived from bone marrow, cord blood, placenta, or mobilized peripheral blood; and
treating the source cells with a metabolic regulator, the metabolic regulator configured to inhibit a tricarboxylic acid cycle of the source cells.

31. The method according to claim 30, wherein the metabolic regulator is configured to inhibit uptake of pyruvate into mitochondria.

32. The method according to claim 30, wherein the metabolic regulator is configured to inhibit conversion of pyruvate to Ac-CoA.

33. The method according to claim 30, wherein the metabolic regulator is configured to inhibit MPC.

34. The method according to claim 33, wherein the metabolic regulator is UK5099.

35. The method according to claim 34, wherein the concentration of UK5099 is at least about 100 nM.

36. The method according to claim 30, wherein the metabolic regulator is configured to inhibit PDH.

37. The method according to claim 36, wherein the metabolic regulator is 1-Aminoethylphosphinic acid (1-AA).

38. The method according to claim 37 wherein the concentration of 1-Aminoethylphosphinic acid is at least about 4 μM.

39. A metabolic regulator for:
(a) activation of a tricarboxylic acid cycle of source cells for the production of definitive hematopoietic cells, or
(b) inhibition of a tricarboxylic acid cycle of source cells for the production of primitive hematopoietic cells.

40. (canceled)
